SEQUENCE LISTING (3 sequences)

SEQ ID NO: 1 - RNA, 23 nt, Homo sapiens
uacccuguagaaccgaauuugug

SEQ ID NO: 2 - RNA, 23 nt, Homo sapiens
uuaaugcuaaucgugauaggggu

SEQ ID NO: 3 - RNA, 23 nt, Homo sapiens
uguaaacauccuacacucucagc

(19) United States
(12) Patent Application Publication
(10) Pub. No.: US 2015/0018230 A1
(43) Pub. Date: Jan. 15, 2015

(54) PLASMA miRNA SIGNATURE FOR THE ACCURATE DIAGNOSIS OF PANCREATIC DUCTAL ADENOCARCINOMA

(71) Applicant: Indiana University Research and Technology Corp., Indianapolis, IN (US)

(72) Inventors: Murray Korc, Indianapolis, IN (US); A. Jesse Gore, Avon, IN (US); Gregory A. Cote, Zionsville, IN (US); Stuart Sherman, Zionsville, IN (US)

(21) Appl. No.: 14/302,331

(22) Filed: Jun. 11, 2014

Related U.S. Application Data
(60) Provisional application No. 61/833,571, filed on Jun. 11, 2013.
(60) Provisional application No. 61/973,144, filed on Mar. 31, 2014.

(51) Int. Cl. C12Q 1/68

(52) U.S. Cl. CPC C12Q 1/6886; C12Q 2600/178; C12Q 2600/158
USPC 506/9; 435/6.11

(57) ABSTRACT

The differential expression of select miRNA in plasma and bile among patients with PDAC, chronic pancreatitis (CP), and controls were measured. Patients (n=215) with treatment-naïve PDAC (n=77), CP with bile or pancreatic duct pathology (n=67), and controls (n=71) that had been prospectively enrolled in a Pancreatobiliary Disease Biorepository at the time of endoscopic retrograde cholangiopancreatography (ERCP) or endoscopic ultrasound (EUS) were identified. Controls were patients with choledocholithiasis but normal pancreata. The sample was separated into training (n=95) and validation (n=120) cohorts to establish and then test the performance of PDAC Signature Panels in diagnosing PDAC. The training cohort (n=95) included age-matched patients with CP and controls. Panels were derived from the differential expression of 10-candidate miRNA in plasma or bile. Differential expression of miR-10b, -155, and -106b in plasma and bile accurately distinguishes individuals with PDAC from those having CP or normal pancreata.

### PRIORITY CLAIM

This application claims the benefit of U.S. provisional patent application No. 61/833,571 filed on Jun. 11, 2013 and U.S. provisional patent application No. 61/973,144 field on Mar. 31, 2014, each of these provisional patent applications are hereby incorporated by reference in its entirety as if each were incorporated individually in its entirety.

### STATEMENT OF GOVERNMENTAL RIGHTS

This invention was made with government support under CA075059 and DK095148 awarded by the National Institutes of Health. The government has certain rights in the invention.

### FIELD OF THE INVENTION

Some aspects of this invention relate to methods for diagnosing pancreatic cancer which makes use of select miRNA levels in sample of peripheral blood, bile, or pancreatic juice to diagnose and treat specific pancreatic cancers.

### SEQUENCE LISTING

miR-10b, Homo sapien.
SEQ. ID. NO. 1:
UACCCUGUAGAACCGAAUUUGUG
miR-155, Homo sapien.
SEQ. ID. NO. 2:
UUAAUGCUAAUCGUGAUAGGGGU
mIR-30C, Homo sapien.
SEQ. ID. NO. 3:
UGUAAACAUCCUACACUCUCAGC

### BRIEF DESCRIPTION OF THE FIGURES

FIGS. 1A-F. Graph illustrating that levels of miR-10b are significantly elevated in plasma of PDAC patients.

FIG. 1A. Plasma miR-10b levels, compared to plasma from normal controls (n=20; closed circles) and chronic pancreatitis patients (CP; n5; gray circles), miR-10b levels are significantly elevated (p<0.001) in the plasma of PDAC patients (n=18; open circles). Horizontal bars denote mean expression levels.

FIG. 1B. Gene expression. qRT-PCR of indicated mRNAs from PANC-1 cells transfected with control (solid bars) or precursor miR-10b (open bars) confirmed the array results.

FIG. 1C. TIP30 levels. qRT-PCR was used to determine TIP30 mRNA levels in PANC-1 cells transfected with control (solid bars) or precursor miR-10b (open bars) for indicated times.

FIG. 1D. TIP30 immunoblotting.

FIG. 1E. Luciferase reporter constructs. The reporter constructs encoded wild type (WT) TIP30 3′UTR and a mutant (M) TIP30 3′UTR in which 6 nucleotides in the binding site were replaced by the paired nucleotides.

FIG. 1F. Luciferase readout. PANC-1 cells transfected with control (solid bars) or precursor miR-10b (open bars) for 20 h and with the wild-type (3′UTR WT) TIP30 3′UTR luciferase construct or the mutant construct (3′UTR M).

FIGS. 2A-D. Graph of data illustrating that miR-10b overexpression and TIP30 downregulation enhance invasion.

FIG. 2A. Effects of miR-10b, EGF, and TGF-f31 on invasion assay in COLO-357.

FIG. 2B. PANC-1 cells were transfected with control (solid bars) or miR-10b (open bars) pre-cursors, and plated (5×104 cells/well) in a Matrigel-coated Boyden chamber.

FIG. 2C. TIP30 silencing. PANC-1 cells were transfected with scrambled control, or two siRNAs (5 and 6) targeting TIP30. Cells (5×104 cells per well) were then plated for Matrigel invasion assays in the absence or presence of EGF (1 nM) for 20 h.

FIG. 2D. Effects of mutant TIP30 on miR-10b-enhanced invasion. PANC-1 cells were transfected for 48 h with control or precursor miR-10b in combination with either empty pCMV-SPORT6(Empty) or pCMV-SPORT6-TIP30 carrying a mutated TIP30 cDNA(TIP30 Mutant) that is resistant to miR-10b induced downregulation.

FIGS. 3A-B show the effects of receptor kinase and pathway inhibitors on invasion.

FIG. 3A. Effects of EGFR inhibitor erlotinib and downstream inhibitors on invasion. COLO-357 and PANC-1 cells were transfected with precursor miR-10b precursor were incubated for 20 h with mM EGF in the absence or presence of 2 μM erlortinib, 10 μM LY294002 (PI3K inhibitor) or 1 μM UO126 (MEK inhibitor).

FIG. 3B. Effects of erlotinib and TβR1 inhibition with SB505124 on invasion.

FIGS. 4A-C shows the effects of miR-10b overexpression on EGF and TGF-β signaling.

FIG. 4A. Effects of miR-10b overexpression on EGFR signaling. PANC-1 cells transfected with control or miR-10b precursor were incubated in the absence of presence of 1 nM EGF for the indicated times.

FIG. 4B. Effects of miR-10b overexpression on EGF and TGF-β signaling. PANC-1 cells transfected with control or miR-10b precursor were incubated in the absence or presence of 1 nM EGF (E), 0.5 nM TGF-β1 (T) or both EGF and TGF-β1 (E+T) for the indicated times.

FIG. 4C. Effects of TIP30 silencing on EGFR signaling. PANC-1 cells transfected with control or siRNA 6 against TIP30 were incubated in the absence or presence of 1 nM EGF for the indicated times.

FIGS. 5A-B shows the effects of miR-10b on expression of genes implicated in EMT. (a-b) qRT-PCR. Control (solid bars) or miR-10b precursor (open bars) transfected COLO-357 and PANC-1 cells were incubated in the absence (serum free: SF) or presence of EGF (E, 1 nM), TGF-β1 (T, 0.5 nM), or both EGF and TGF-31 (E+T) for 24 h and then harvested for RNA extraction and qRT-PCR for the indicated mRNAs.

FIG. 6. Study population (n=215). PDAC=pancreatic ductal adenocarcinoma; CP=chronic pancreatitis. Seven subjects with PDAC have missing stage information in the training (n=1) and validation (n=6) cohorts.

FIGS. 7A-E. miRNA expression in plasma and bile samples from the training cohort (n=95). IQR=interquartile range; y axis=relative expression of miRNA Box plot for each of the 10 miRNA measured in plasma and bile from the training cohort.

FIG. 7A. Upper panel miR-10b, lower panel miR-196a.

FIG. 7B. Upper panel miR-106b, lower panel miR-181a.

FIG. 7C. Upper panel miR-212, lower panel miR-181b.

FIG. 7D. Upper panel miR-155, lower panel miR-132.

FIG. 7E. Upper panel miR-30c, lower panel miR-21.

FIGS. 8A-C. miRNA expression in plasma and bile from the validation cohort (n=120). IQR=interquartile range; y axis=relative expression of miRNA. Box plot for each of the 5 miRNA measured in plasma and bile from the validation cohort.

FIG. 8A. mir-10b

FIG. 8B. Upper panel miR-106b, lower panel miR-212.

FIG. 8C. Upper panel miR-155, lower panel miR-30c.

FIG. 9. Chart illustrating the Fold Change in miR-10b measured in patients diagnosed with pancreatic cancer before (Pre-SX) and after (Post-SX) the surgical removal of pancreatic tumors.

FIG. 10. Graph illustrating the Fold Change in miR-10b measured in patients diagnosed with pancreatic cancer before (Pre-SX) and after (Post-SX) the surgical removal of pancreatic tumors.

### BACKGROUND/SUMMARY

The absence of reliable blood markers for pancreatic ductal adenocarcinoma (PDAC) reduces the potential effectiveness of screening strategies in at-risk populations such as those with chronic pancreatitis (CP). Furthermore, among individuals with PDAC, there are no histological or molecular targets currently applied in the clinical setting to direct therapy or inform survival estimates. Therefore, the discovery of biomarkers derived from blood or bile that facilitate the distinction of PDAC from CP and provide prognostic information for individuals with PDAC would greatly impact patient management. Additionally, biomarkers may guide studies designed to elucidate dysplasia-to-carcinoma mechanism(s) specific to PDAC and identify gene or protein targets for novel therapies.

An aspect of the present disclosure relates to a method of detecting the presence of pancreatic cancer in a patient, comprising obtaining a sample of peripheral blood from a patient and assaying the sample of peripheral blood for the presence of miR-10b and determining that the patient has PDAC if the level of miR-10b is elevated.

A PDAC Signature Panel developed from the differential expression of miRNA in plasma, bile or pancreatic juice represents a promising and novel diagnostic test for PDAC. This Panel accurately distinguishes individuals with PDAC from those with CP, an at-risk population who may be considered for screening, and has substantial potential to improve the diagnostic accuracy of ERCP for indeterminate bile duct strictures. Future studies are needed to validate the diagnostic accuracy of this Panel in larger cohorts, as well as to assess their usefulness as markers that correlate with survival and response to therapy for individuals with PDAC.

Accurate peripheral markers for the diagnosis of pancreatic ductal adenocarcinoma (PDAC) are lacking. The differential expression of select miRNA in plasma and bile among patients with PDAC, chronic pancreatitis (CP), and controls were measured.

Patients (n=215) with treatment-naïve PDAC (n=77), CP with bile or pancreatic duct pathology (n=67), and controls (n=71) that had been prospectively enrolled in a Pancreatobiliary Disease Biorepository at the time of endoscopic retrograde cholangiopancreatography (ERCP) or endoscopic ultrasound (EUS) were identified. Controls were patients with choledocholithiasis but normal pancreata.

The sample was separated into training (n=95) and validation (n=120) cohorts to establish and then test the performance of PDAC Signature Panels in diagnosing PDAC. The training cohort (n=95) included age-matched patients with CP and controls. Panels were derived from the differential expression of 10-candidate miRNA in plasma or bile. miRNA having excellent accuracy (area under curve>0.90) for inclusion in regression models for each medium were selected.

Using the training cohort, the differential expression of 9/10 miRNA in plasma (-10b, -30c, -106b, -132, -155, -181a, -181b, -196a, -212) and 7/10 in bile (excluding miR-21, -132, -181b). Of these, five (-10b, -155, -106b, -30c, -212) had excellent accuracy for distinguishing PDAC were confirmed. In the training and validation cohorts, the sensitivity/specificity for a PDAC Panel derived from plasma was 95%/100% and 100%/100%, respectively; in bile, these were 96%/100% and 100%/100%. Differential expression of miR-10b, -155, and -106b in plasma and bile accurately distinguishes individuals with PDAC from those having CP or normal pancreata.

Some embodiments include methods for treating a patient, comprising the steps of: contacting a portion of plasma from a patient with at least one probe specific for at least one miRNA selected from the group consisting of miR-10b, and miR-106b; using a signal produced by the contacting step to determine a level of at least one miRNA selected from the group consisting of: miR-10b, and miR-106b, in the sample; comparing the level of the at least one miRNA measured in the sample to a statistically validated threshold for miR-10b, and/or miR-106b, levels; and concluding that the patient is positive for pancreatic ductal adenocarcinoma if the threshold for miR-10b in the sample is greater than about 3.579, or the threshold of miR-106b in the sample is greater than about 2.920.

In some embodiments the contacting step further includes contacting the sample with at least one probe for at least one miRNA, selected from the group consisting of: miR-21, miR-30c, miR-132, miR-155, miR-181a, miR-181b, miR-196a, and miR-212.

In some embodiment a patient is concluded to be positive for pancreatic ductal adenocarcinoma if the threshold of miR-10b measured in the sample is greater than about 3.579, and the threshold of miR-106b measured in the sample is greater than about 2.920.

In some embodiment the method includes the step of generating cDNA from RNA present in the sample before conducting the contacting step or as part of the contacting step. In some embodiments the methods further including the step of: recommending that the patient be treated for pancreatic ductal adenocarcinoma if the concluding step is positive for pancreatic ductal adenocarcinoma.

Some embodiments include a method of treating a patient that includes contacting a portion of bile from a patient with at least one probe specific for at least one miRNA selected from the group consisting of miR-10b, miR-106b, miR-155, and miR-212; using a signal produced by the contacting step to determine a level of at least one miRNA selected from the group consisting of: miR-10b, miR-106b, miR-155, and miR-212 in the sample; comparing the level of the at least one miRNA measured in the sample to a statistically validated threshold for miR-10b, miR-106b, miR-155, and/or miR-212 levels; and concluding that the patient is positive for pancreatic ductal adenocarcinoma if the threshold of miR-10b in the sample is greater than about 3.497, or the threshold of miR-106b in the sample is greater than about 5.261, or the threshold of miR-155 in the sample is greater than about 5.232, or the threshold of miR-212 in the sample is greater than about 4.163.

In some embodiments the method of using bile to determine if a patient is positive for a form of pancreatic cancer includes the step of identifying that a patient is positive for a cancer if the threshold of miR-10b in the sample is greater than about 3.497, the threshold of miR-106b in the sample is greater than about 5.261, the threshold of miR-155 in the sample is greater than about 5.232, and the threshold of miR-212 in the sample is greater than about 4.163.

Some embodiments include contacting a portion of pancreatic juice from a patient with at least one probe specific for at least one miRNA selected from the group consisting of miR-155, miR-106b, miR-10b, miR-212, and miR-30C; using a signal produced by the contacting step to determine a level of at least one miRNA selected from the group consisting of: miR-155, miR-106b, miR-10b, miR-212, and miR-30C in the sample; comparing the level of the at least one miRNA measured in the sample to a statistically validated threshold for miR-155, miR-106b, miR-10b, miR-212, and/or miR-30C levels; and concluding that the patient is positive for pancreatic ductal adenocarcinoma if the threshold of miR-155 in the sample is greater than about 4.42, or the threshold of miR-106b in the sample is greater than about 4.54, or the threshold of miR-10b in the sample is greater than about 4.54, or the threshold of miR-212 in the sample is greater than about 3.69 or the threshold of miR-30 measured in the sample is greater than 3.27. In some embodiments a patient is determined to be positive for pancreatic ductal adenocarcinoma if the threshold of miR-155 in the sample is greater than about 4.42, the threshold of miR-106b in the sample is greater than about 4.54, the threshold of miR-10b in the sample is greater than about 4.54, the threshold of miR-212 in the sample is greater than about 3.69 and the threshold of miR-30 measured in the sample is greater than 3.27.

In some embodiments the method includes creating cDNA from miRNA in a sample of fluid or tissue from a patient and probing the sample for the presence of specific miRNAs whose levels are changed in patients that have a form of pancreatic cancer relative to patients that do not have any form or pancreatic cancer of a different from of pancreatic cancer. In some embodiments the method further includes at least one step of preparing a sample of blood, bile, or pancreatic juices such as deriving plasma from a sample of peripheral blood from a patient prior to contacting the processed sample with a probe specific for certain indicia of specific miRNAs. In some embodiments, miRNA's can be used to distinguish between patients with chronic pancreatitis (CP) and patients with PADC. In other embodiments, miRNA expression levels are normalized with an internal standard to miRNA's present in the samples, wherein these particular miRNA's are expressed at similar levels in patients with pancreatic cancer, for example PDAC, and in patients without pancreatic cancer. In some embodiments, this internal standard is miR-425-5p.

### DESCRIPTION

As used herein, unless explicitly stated otherwise the term ‘treating’ may include screening, diagnosing, selecting, identifying, test and the like a patient to determine if a patient has or is at a heightened or reduced risk for developing a specific condition and/or disease. The term treating also includes providing a given patient with therapy including surgical and or medicinal interventions, including for example chemo and/or radio therapy.

In some embodiments the assay includes subjection of at least a portion of the sample of peripheral blood of a patient to quantitative reverse transcription (RT) PCR. In a further embodiment that quantitative RT PCR includes a miR-10b specific primer. Quantitative RT PCR methods are well known in the art. In some embodiments the sample may be plasma, in other embodiments the sample may be bile, and in still other embodiments the sample may be pancreatic juice,

As used herein, “patient” or “subject” means an individual having symptoms of, or at risk for, pancreatic cancer or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the methods contemplated herein.

For the convenience of the reading, some of the acronyms used in this application are defined as follows: PDAC Pancreatic ductal adenocarcinoma; CP Chronic pancreatitis; miRNA micro RNA; FNA Fine needle aspirate; EUS Endoscopic ultrasound; ERCP Endoscopic retrograde cholangiopancreatography; Ct Cycle threshold; IQR Interquartile range; ROC Receiver operating curve; AUC Area under the curve; PanIN pancreatic intraepithelial neoplasia; IPMN intraductal papillary mucinous neoplasm; ISH in situ hybridization; Ago2 Argonaute2.

Due to their biological stability and role in cancer pathobiology, [1, 2, 3] microRNAs (miRNAs) have substantial potential as cancer biomarkers.[4, 5] miRNAs are short, non-coding RNAs consisting of 18-25 nucleotides that function by targeting specific mRNA moieties for translational repression or degradation, thereby regulating several biological processes including cell proliferation, migration, invasion, survival and metastasis.[6, 7, 8, 9] miRNAs have also been implicated in the modulation of PDAC progression and patient survival.[10, 11, 12, 13, 14, 15] The majority of studies evaluating miRNAs in PDAC are derived from surgical tissue samples; nearly 100 miRNAs have been identified by their differential expression in PDAC tissue.[10, 11, 12, 13, 14, 15, 16, 17] For maximal utility in the clinical setting—screening, treatment planning, and assessment of treatment response—the optimal PDAC biomarker(s) would be identified from a blood sample, and following referral for specialized endoscopic procedures, biomarkers could be readily assayed in bile or pancreatic juice aspirates, or PDAC tissue obtained by fine needle aspirate (FNA).

As disclosed herein, the utility of select miRNAs as diagnostic markers of PDAC, by comparing the differential expression of miRNA in plasma and bile among individuals with PDAC, CP, and normal pancreas was assessed. Secondarily, the differential expression of miRNA in the subgroup of individuals having pancreatic juice samples available for analysis was determined.

### Materials and Methods

Cell culture. ASPC-1 and PANG-I human PCCs were obtained from ATCC (Manassas, Va.), which validated their authenticity. COLO-357 and T3M4 human PCCs were a gift from Dr. R S Metzger at Duke University. Their authenticity was validated by chromosomal analysis. ASPC-1 cells were grown in RPMI 1640, and PANC-1 and COLO-357 cells were grown in DMEM. Media were supplemented with 5% fetal bovine serum, 100 U/ml penicillin and 100 μg/ml streptomycin (complete medium) at 37° C. in humidified 5% CO2 incubator. Serum-free medium (SF) was supplemented with 0.1% bovine serum albumin, 5 μg/mL apotransferrin, and 5 ng/mL sodium selenite. EGF (Millipore, Billerica, Mass.) and TGF-β (Genentech, Inc., South San Francisco, Calif.) were added at the indicated concentrations.

RNA Isolation and Quantitation. Total RNA was extracted using TRIZOL reagent (Invitrogen, Carlsbad, Calif.). Assaying of mature miRNAs was performed using TaqMan miRNA qRT-PCR kit (Life Technologies, Gaithersburg, Md.) using RNU6B as an internal control. To assay mRNAs, RNA (500 ng) was reverse transcribed to cDNA with a high capacity RNA-to-cDNA master mix and qRT-PCR was performed with Power SYBR® Green Master Mix (both from Life Technologies, Gaithersburg, Md.). Primers were designed using Prime-BLAST online tool.

Transfection. Cells were transfected with 20-60 nM miR-10b precursor (known as pre-miR has-miR-10b precursor, Life Technologies, Gaithersburg, Md.), or a control precursor (known as pre-miR non-targeting negative control) at 50% confluency. Transfected cells were plated for MTT assay (24 h post-transfection) or transwell invasion assays (48 h post-transfection). Non-targeting siRNA or siRNAs, (ON-TARGETplus siRNA, Dharmacon, Lafayette, Colo.), designed to target TIP30 were used to silence TIP30 in COLO-357 (60 nM) and PANG-I (20 nM) cells. For TIP30 rescue experiments, PANC-1 cells were transfected with 20 nM miR precursor and 250 ng of pCMV-SPORT6-TIP30 cDNA and Lipofectamine 2000 (both from Invitrogen, Carlsbad, Calif.), which was used in all transfection studies.

MTT assay. Cells were plated in 96-well plates (6 triplicates per sample), allowed to grow for 72 h in complete medium before adding 3-(4,5-dimethylthiazol-2-yl)-2,5-diphenyltetrazolium bromide (MTT, Sigma-Aldrich, St Louis, Mo.) for 4 h. Absorbance was measured at 570 nm.

Invasion and Migration Assays. miR-10b precursor and precursor control transfected cells (5×104) were plated in Matrigel pre-coated transwell chambers (BD Biosciences, San Jose, Calif.) and assays performed as previously reported. UO126 (1 μM) were from Alexis Biochemicals (San Diego, Calif.). Erlotinib (2 μM; Genentech, Inc., South San Francisco, Calif.), and LY294002 (10 μM; Calbiochem, San Diego, Calif.), were added to SF in the lower chamber, in the absence or presence of 1 nM EGF and/or 0.5 nM TGF-β1. Wound healing assays were performed as previously reported. Briefly, 24 hours after cell transfection with pre-miR-10b oligonucleotides, cells were serum starved overnight, and the monolayer was scratched with a 200 μL pipette tip generating two parallel wounds. Cells were then rinsed twice with HBSS and incubated for 24 h in SF media in the absence or presence of 1 nM EGF, 0.5 nM TGF-β1, or their combination, and migration was calculated as the percent change in the wound area.

Microarray and data analysis. miR-10b precursor or control transfected PANC-1 cells were harvested at 20 h post transfection. Total RNA (three replicates per condition) was extracted using TRIZOL reagent. Microarray analysis for microRNAs was performed by the Genomics and Microarray Core Facility at Dartmouth Medical School. Array data were registered with GEO (accession # GSE40189) for public access.

Immunoblotting. Whole cell extracts were prepared by lysis in ice-cold lysis buffer (20 mM Tris-HCl (pH 7.5), 150 mM NaCl, 1 mM EDTA, 1 mM EGTA, 1% Triton, 2.5 mM sodium pyrophosphate, 1 mM beta-glycerophosphate, 1 mM Na3VO4, 1 mM PMSF, and 1 tablet of complete protease inhibitor/10 ml lysis buffer). Lysates (25 μg/lane) were subjected to 10-12% SDS-PAGE and transferred to PVDF (Millipore, Billerica, Mass.), and immunoblotted. Antibodies directed against the following antigens were used: EGFR, Phospho-EGFR Tyr1148, phospho-AKT Ser473, AKT, phospho-p44/42 MAPK Thr202/Tyr204, p44/42 MAPK, phospho-p38 MAPK Thr180/Tyr182, p38 MAPK (all from Cell Signaling Technology, Danvers, Mass.); ERK-2(C-14, Santa Cruz Biotechnology, Santa Cruz, Calif.).

Constructs. The LightSwitch TIP30 3′UTR Reporter (Switchgear Genomics, Menlo Park, Calif.) was utilized to assess whether there is a direct interaction between miR-10b and TIP30 3′UTR. Sited-directed mutagenesis kits (Stratagene, La Jolla, Calif.) were used to introduce a six base pair mutation in the seed-binding site of TIP30 3′UTR. The pCMV-PORT6 TIP30 cDNA construct used in the TIP30 rescue experiment was purchased from Open Biosystems, Huntsville, Ala.

Luciferase Reporter Assay. Cells in 24-well plates (50% confluency) were co-transfected with miR-10b precursor (20 nM) and TIP30 3′UTR Reporter (200 ng). Cell extracts were prepared 24 h later, and luciferase activity was measured using the Dual-Glo luciferase assay system (Promega, Fitchburg, Wis.).

Circulating miR-10b. Quantitation Plasma from 20 normal, 5 chronic pancreatitis and 17 PDAC patients was obtained from the Indiana University Simon Cancer Center Solid Tissue Bank (Indianapolis, Ind.). RNA was isolated from 100 μl of plasma using Trizol LS (Life Technologies, Carlsbad, Calif.) according to the manufacturers' recommendations. Quantitative reverse transcription PCR (qRT-PCR) for miR-10b was performed with a Taqman micro RNA reverse transcription kit and a miR-10b-specific primer/probe set (Life Technologies). Each plasma sample was assayed in duplicate, and Ct values were obtained using a Viia 7 Real-time PCR System (Life Technologies). miR-10b levels were normalized to miR-16 levels and fold increases were calculated as described.

Statistical Analysis. Results are presented as mean±SEM. Statistical differences between groups were assessed using one sample T-test analysis, one-way or two-way ANOVA, as indicated, followed by Bonferroni post-test analysis. P<0.05 was considered as statistically significant.

miR-10b is Upregulated in PDAC Peripheral Blood

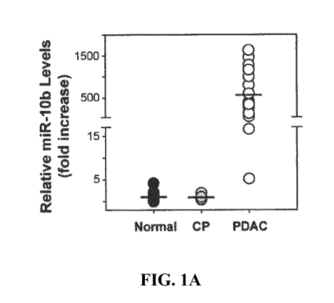

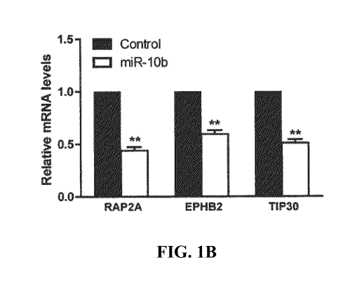

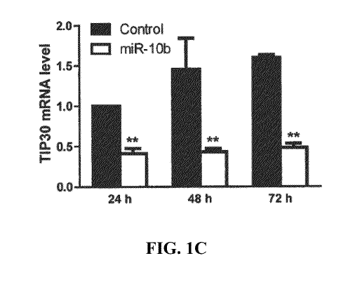

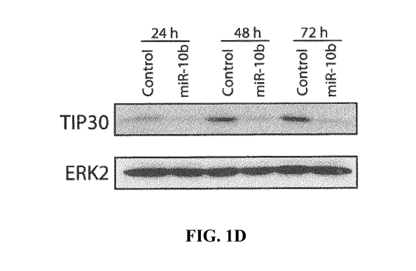

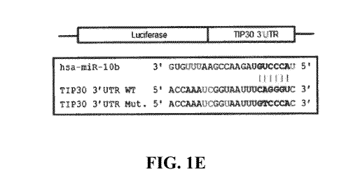

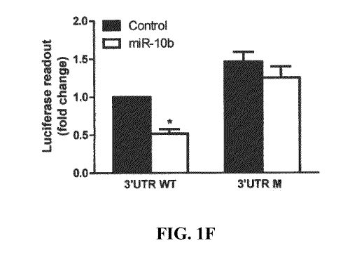

miR-10b is expressed at high levels in the cancer cells in PDAC, and several studies have implicated miR-10b in cancer metastasis. To determine if miR-10b expression is increased in PDAC, the miR-10b levels in the plasma of 20 normal controls, 5 patients with chronic pancreatitis, and 17 PDAC patients were measured. Referring now to FIG. 1, data in FIGS. 1A, 1B and 1C, are the means±SEM from three experiments, analyzed by two-way ANOVA followed by Bonferroni adjustment. *p<0.01 and **p<0.001 compared with its respective controls. Levels of miR-10b were increased 575-fold in PDAC patients by comparison with the corresponding levels in either normal controls (P<0.001) or patients with chronic pancreatitis (P<0.001; FIG. 1A).

Since the metastatic process is preceded by invasion, invasion associated miR-10b targets that could lead to enhanced metastasis in PDAC were examined. Accordingly, microarray studies were conducted using RNA from PANC-1 cells transfected with a miR-10b precursor. Fifty-five genes were downregulated by at least 40% upon miR-10b overexpression (data not shown), and 3 of these genes (RAP2A, EPHB2, and TIP30) affect pathways that have the potential to suppress cancer cell invasion. Quantitative reverse transcription PCR (qRT-PCR) confirmed that the mRNA levels of all three genes were significantly decreased in PANC-1 cells expressing high level of miR-10b compared to respective controls (FIG. 1B). In silico analysis with miRanda, PicTar and TargetScan computational tools for miRNA target prediction confirmed that TIP30, RAP2A and EPHB2 were potential miR-10b targets.

TIP30 (Tat-interacting protein 30), also known as HIV-1 Tat interactive protein 2 (HTATIP2) or CC3, was of particular interest because it suppresses metastases in lung, breast, and hepatocellular cancers and regulates EGF-induced EGFR endocytosis. Given the important role of EGFR in PDAC, miR-10b was tested to determine if it down regulates TIP30 mRNA. Accordingly, qRT-PCR and immunoblotting were conducted using RNA and protein extracted from miR-10b-overexpressing PANC-1 cells. Referring now to FIG. 1C, a highly specific anti-TIP 30 antibody was used in immunoblotting analysis, revealing a marked decrease in TIP30 protein levels in the presence of miR-10b by comparison with control. Increased miR-10b expression led to decreased TIP30 mRNA (FIG. 1C) and protein (FIG. 1D) levels at 24, 48 and 72 h post-transfection. A similar decrease in TIP30 protein levels was seen in ASPC-1, COLO-357, and T3M4 cells following transfection with miR-10b precursor, indicating that this downregulation was not unique to PANC-1 cells. Moreover, transfection with the miR-10a precursor, which shares the same sequence with miR-10b except for a difference of one nucleotide in the non-seed region, resulted in variable but reproducible decreases in TIP30 levels in all four PCCs.

To confirm that TIP30 is a direct target of miR-10b, a TIP30 3′UTR luciferase construct in and a mutant construct, in which 6 nucleotides in the potential binding site were replaced, was used (FIG. 1E). Referring now to FIG. 1F. Co-transfection of PANC-1 cells with the TIP30 3′UTR construct and miR-10b precursor caused a 50% decrease in luciferase activity compared with the negative control, and this suppression of TIP30 expression was completely reversed by the six-nucleotide substitution in the core binding site.

miR-10b Enhances Pancreatic Cancer Cell Invasion

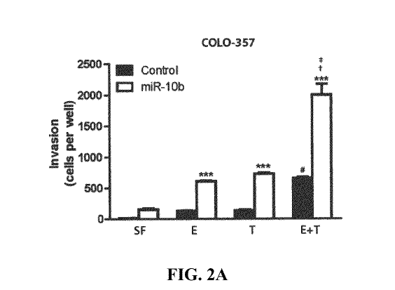

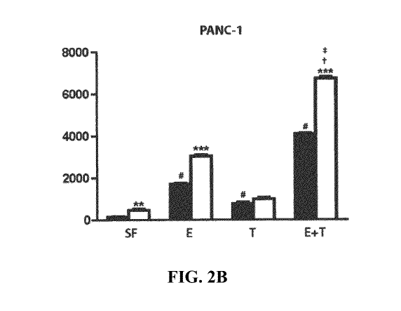

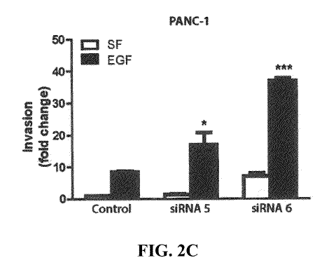

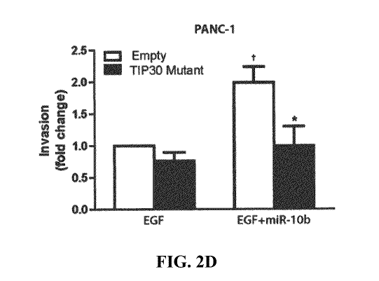

To determine whether miR-10b enhances human PCC invasion, mir-10b precursor was transfected into two human PCCs. In PANC-1 and COLO-357 cells, EGF (1 nM) or TGF-β1 (0.5 nM) enhanced invasion, and their combined action was greater than that of either growth factor alone (FIGS. 2A-B). Referring to FIG. 2B, invasion was determined after incubating cells for 20 h in the absence (serum free: SF) or presence of EGF (E, 1 nM). TGF-β1 (T, 0.5 nM), or both EGF and TGF-β1 (E+T). Cells that had invaded across the membrane were stained and counted. Effects of mutant TIP30 on miR-10b-enhanced invasion. PANC-1 cells were transfected for 48 h with control or precursor miR-10b in combination with either empty pCMV-SPORT6(Empty) or pCMV-SPORT6-TIP30 carrying a mutated TIP30 cDNA(TIP30 Mutant) that is resistant to miR-10b induced downregulation.

Overexpression of miR-10b increased invasion in PANC-1 cells, and enhanced EGF-mediated invasion in both PCCs and TGF-β1-mediated invasion in COLO-357 cells. Moreover, the combination of both growth factors exerted a significantly (p<0.005) greater stimulatory effect on invasion in cells with high miR-10b levels (FIG. 2A-B), which is potentially important clinically given that miR-10b, EGFR, and TGF-β are all overexpressed in PDAC.

To determine whether TIP30 knockdown mimics miR-10b actions on PCC invasion, PANC-1 cells were transfected with two distinct siRNAs targeting TIP30, in the absence or presence of 1 nM EGF for 20 h. Referring now to FIG. 2C, TIP30 silencing by either siRNA resulted in increased EGF-stimulated invasion, indicating that TIP30 is a negative regulator of PCC invasion. To confirm that TIP30 downregulation was essential for miR-10b-induced stimulation of cell invasion, the pCMV-SPORT6-TIP30 vector, which encodes a TIP30 cDNA that is not regulated by miR-10b due to a mutated 3′UTR binding site was used. Accordingly, experiments were carried out in the presence of 1 nM EGF, in the absence or presence of transfected miR-10b using cells expressing empty vector or the pCMV-SPORT6-TIP30 vector. This experimental design allowed for the specific evaluation of the consequence of expressing a TIP30 that was resistant to miR-10b downregulation.

Referring now to FIG. 2D, the effects on invasion by 1 nM EGF were then determined. Data in each panel are the means±SEM from three experiments, and were analyzed by two-way ANOVA followed by Bonferroni adjustment. *p<0.05, **p<0.01, and ***p<0.001 compared with respective controls; ^{#}p<0.05 compared with corresponding control SF; ^{†}p<0.01 compared with EGF alone and ^{‡}p<0.01 compared with TGF-β1 alone in cells transfected with miR-10b precursor; ^{†}p<0.05 compared with empty pCMV-SPORT6, and *p<0.05 compared with pCMV-SPORT6-TIP30 in miR-10b-transfected group. When pCMV-SPORT6-TIP30 was transfected into PANC-1 cells, the stimulatory effect of miR-10b overexpression on EGF-induced invasion was completely abrogated. Thus, TIP30 is a functional target of miR-10b whose downregulation promotes EGF-induced invasion.

miR-10b Promotes EGFR-TGF-β Interactions that Enhance Invasion

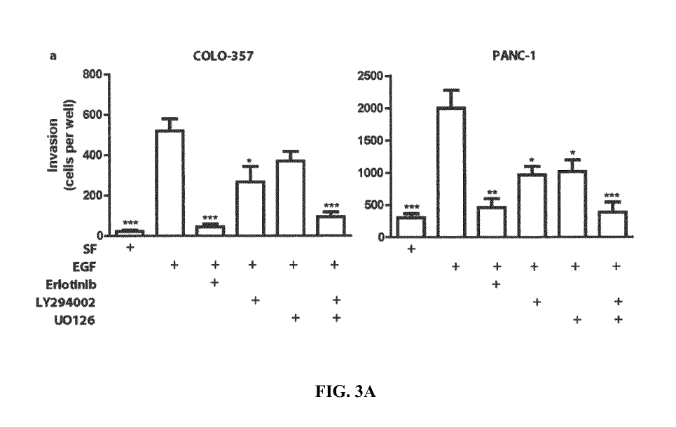

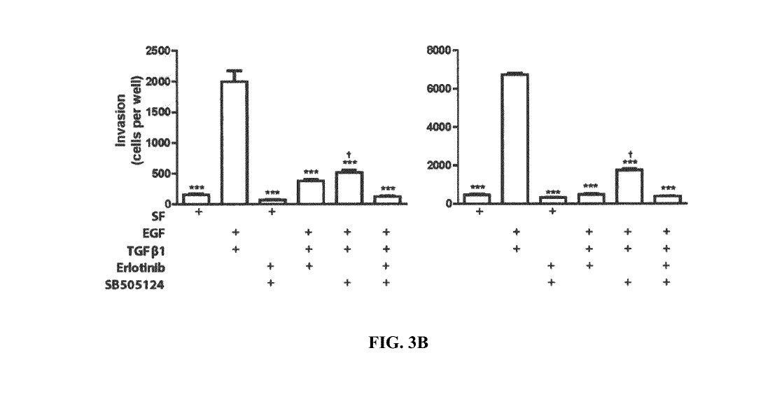

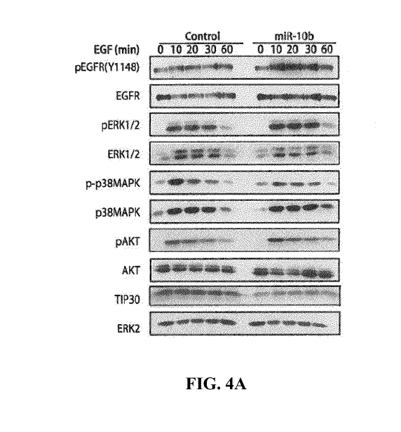

Specific inhibitors were used next to confirm that EGF and TGF-β acted through their respective receptors with respect to their individual and combined stimulatory effects on invasion in the presence of miR-10b. Referring now to FIG. 3A. The EGFR inhibitor erlotinib (2 μM) completely blocked EGF-induced invasion in the presence of miR-10b in both COLO-357 and PANC-1 cells. Still referring to FIG. 3A. Although the PI3K inhibitor LY294002 (10 μM) and the MEK inhibitor UO126 (1 μM) partially blocked this effect, the combination of LY294002 and UO126 were as effective as erlotinib in suppressing EGF-mediated invasion. Referring now to FIG. 3B. Moreover, miR-10b's effects on combined EGF-TGF-β1 actions on invasion were markedly attenuated by EGFR kinase inhibition with erlotinib and TβR1 kinase inhibition with SB505124, and were completely abrogated by their combined actions, indicating that EGF and TGF-β1 act through their respective receptors to cross-talk and dramatically enhance invasion. COLO-357 and PANC-1 cells transfected with precursor miR-10b were incubated for 20 h with 1 nM EGF, 0.5 nM TGF-β1 and, erlotinib (2 μM) or SB505124 (10 μM). and effects on invasion were determined. Data in all panels are the means±SEM from three experiments, and were analyzed by ANOVA followed by Bonferroni adjustment. In panels (a) and (b), *p<0.05, **p<0.01, and ***p<0.001, compared with EGF alone; in panels (c) and (d)***p<0.001 compared with EGF and TGF-β1 combination, and ^{†}p<0.05 compared with corresponding serum free (SF) values.

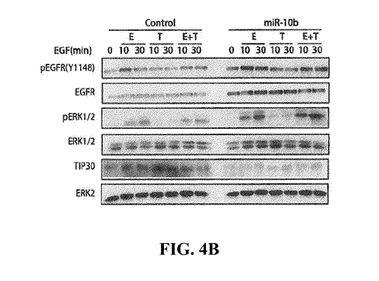

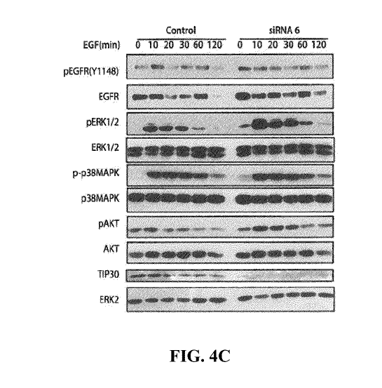

TIP30 is a crucial component of the complex regulating endocytotic trafficking of EGFR in breast and liver cancer cells and may act to prolong downstream signaling. It was sought to determine whether miR-10b modulates EGFR protein and/or mRNA levels and EGFR signaling in PANC-1 cells. In control cells, EGF caused a gradual decrease in EGFR protein levels and this effect was attenuated in miR-10b overexpressing cells (FIG. 4A) but was not associated with any alteration in EGFR mRNA levels. EGF also rapidly increased EGFR phosphorylation (Tyr 1148), as well as ERK1/2 (Thr-202/Tyr-204). Both of these effects were enhanced by miR-10b (FIG. 4A). Referring now to FIG. 4A. By contrast, EGF-induced AKT phosphorylation (Ser 473) and p38 MAPK phosphorylation (Thr-180/Tyr-182) were not altered by miR-10b.

To determine whether TGF-β1 modulated the actions of miR-10b on EGF-induced FGFR and ERK phosphorylation, experiments were repeated in the absence or presence of EGF (1 nM) and TGF-β1 (0.5 nM). TGF-β1 alone did not induce EGFR or ERK phosphorylation, and did not alter the actions of EGF on EGFR and ERK phosphorylation, irrespective of the presence or absence of high levels of miR-10b (FIG. 4B).

To assess whether downregulation of TIP30 alone is sufficient to modulate EGFR signaling in PCCs, TIP30 levels were suppressed with a specific sIRNA. Referring now to FIG. 4C. TIP30 knockdown attenuated EGF-induced EGFR downregulation and enhanced EGFR and pERK1/2 phosphorylation without altering p38 MAPK phosphorylation. Referring now to FIG. 4C. However, in contrast to the actions of miR-10b, siRNA targeting of TIP30 enhanced EGF-induced AKT phosphorylation (FIG. 4C). In FIGS. 4A-C, immunoblotting for the indicated proteins was then carried out; ERK2 was used to assess loading of lanes.

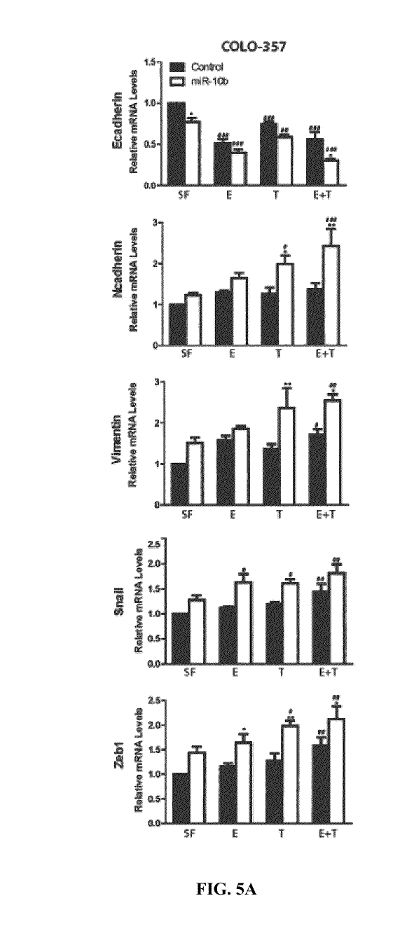

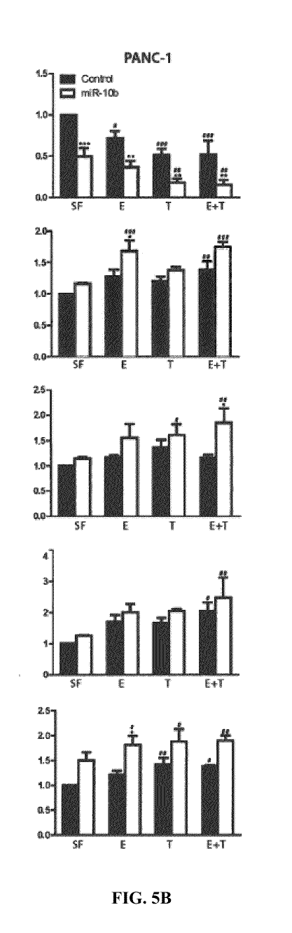

Cancer cell invasion is often associated with enhanced EMT, and TGF-β1 is a well-known EMT inducer in PCCs. Whether miR-10b modulated the effects of TGF-β and EGF on the expression of EMT-associated genes was determined. Referring now to FIG. 5. Data are the means±SEM from four experiments. Two-way ANOVA was performed followed by Bonferroni adjustment. *p<0.05, **p<0.01, and ***p<0.001 compared with respective control transfected cells; #p<0.05, ##p<0.01, and ###p<0.001 compared with respective cells in SF condition. In both COLO-357 and PANC-1 cells, miR-10b significantly decreased E-cadherin levels, and this effect was most pronounced in the presence of the combination of TGF-β1 and EGF (FIG. 5). In both cell lines, EGF plus TGF-β1 increased N-cadherin and vimentin expression, but only in the presence of high miR-10b levels. Moreover, in the presence of miR-10b, N-cadherin expression was increased by TGF-β1 alone in COLO-357 cells, and by EGF alone in PANC-1 cells, where EGF plus TGF-β1 also increased N-cadherin in the absence of added miR-10b. Snail and Zeb1 expression was increased in COLO-357 cells in response to either or both growth factor in the presence of miR-10b, as well as in the presence of both growth factors without added miR-0b. A similar pattern was observed with respect to Zeb1 in PANC-1 cells, except that TGF-β1 was effective even in the absence of added miR-10b. However, PANC-1 cells only upregulated Snail levels in response to both growth factors, and this effect was independent of addition of miR-10b.

Both EMT and invasion are associated with enhanced motility. The effect of miR-10b to modulate PCC migration was tested. In COLO-357 cells, EGF (1 nM), TGF-β1 (0.5 nM), or their combination, significantly increased cell migration, irrespective of the presence or absence of miR-10b, and their combined effect on migration was significantly greater than with either growth factor alone. By contrast, in PANC-1 cells, EGF only enhanced migration following miR-10b transfection, whereas TGF-β1 and the combination of EGF or TGF-β1 enhanced migration in control transfected cells, and this effect was significantly enhanced in the presence of miR-10b. Other supporting evidence regarding the molecular biology and physiology of pancreatic and pancreatic cancer cells can be found in U.S. provisional patent application No. 61/833,571 filed on Jun. 11, 2013 and incorporated herein by reference in its entirety.

Effects of miR-10b on Expression of Several Known miR-10b Target Genes

The effects of miR-10b on the expression of several known miR-10b target genes: HOXD10, TIAMI, KLF4, SDC1, and NF1 was examined. qRT-PCR indicated that the expression of SDC1 and NF1 was decreased by miR-10b (data not shown). miR-10b also inhibited the expression of KLF4 in all four PCCs while it had no effect on the expression of HOXD10 or TIAM1.

miR-10b was initially reported as a metastasis promoter in breast cancer, and subsequently implicated in enhancing metastasis in cancers of the esophagus, lung, kidney, colon, liver, and pancreas, and invasion in glioblastoma. In breast cancer, miR-10b down-regulates HOXD10 leading to the increased expression of the metastasis-promoting RhoC. Moreover, in a syngeneic orthotopic mouse model of breast cancer, miR-10b antagomirs significantly attenuated the frequency of metastases to the lungs.

The present disclosure determined that plasma miR-10b levels are markedly increased in PDAC patients by comparison with either normal subjects or patients with chronic pancreatitis, showing that assaying for miR-10b could be a useful diagnostic biomarker (FIG. 1). Moreover, miR-10b overexpression in PCCs enhanced EGF-stimulated invasion. By gene profiling, immunoblotting, and luciferase reporter assays, TIP30 was identified as a target directly downregulated by miR-10b overexpression. Four lines of evidence showed that miR-10b promoted EGF-mediated invasion by down-regulating TIP30 and enhancing EGFR signaling. First, expression of a TIP30 cDNA resistant to silencing by miR-10b abrogated miR-10b actions on invasion. Second, siRNA-mediated silencing of TIP30 resulted in increased EGF-mediated invasiveness. Third, both high miR-10b levels and TIP30 knockdown enhanced EGF-induced EGFR tyrosine phosphorylation and ERK phosphorylation, while attenuating EGFR down regulation. Fourth, EGF-mediated invasion in the presence of high miR-10b levels was blocked by erlotinib and by the combined actions of the PI3K inhibitor LY294002 and the MEK inhibitor UO126, two key pathways downstream of EGFR activation.

PCCs and PDACs express high levels of EGFR, a known mediator of mitogenic signals in general and a promoter of PCC invasion and metastasis that also is essential for PDAC initiation and progression. PDACs also express high levels of human EGFR 2 (HER2) and HER3, which heterodimerize with EGFR thereby diversifying and amplifying signaling cascades. Moreover, PDACs express high levels of TGF-βs, 14-3-3sigma, MUC1, β1-integrins, and delta-N p63, all of which enhance EGFR's ability to activate aberrant pathways that contribute to the invasiveness of PCCs. In the present disclosure we also determined that TGF-β increased EGF-mediated cell invasion, and that this effect was markedly enhanced by high levels of miR-10b. Impressively, the combination of EGF, TGF-β, and miR-10b induced a marked increase in cancer cell invasion and EMT-like alterations in gene expression. Thus, miR-10b was facilitating deleterious cross-talk between EGF and TGF-β in a manner that promotes PCC invasion. Given that miR-10b, EGFR, and TGF-β are often overexpressed in PDAC, these observations show that suppressing miR-10b may prevent metastasis in PDAC while interrupting deleterious EGF-TGF-β interactions thereby potentially enhancing the therapeutic effectiveness of targeting both pathways.

EGF binding to EGFR activates multiple signaling pathways in PCCs, such as the Ras/Raf/MAPK and Rac/JNKIMAPK-38, and EGF then undergoes rapid endocytosis. The EGF/EGFR complexes initially enter into the early endosomes where the EGFR kinase is still active, and then traffic to the late endosome prior to entering the lysosome where EGFR undergoes degradation. TIP30 accelerates EGFR progression from early to late endosomes, whereas TIP30 silencing causes the retention of EGF-EGFR complexes in the early endosome thereby attenuating EGFR degradation and prolonging its signaling. Inasmuch as PDAC is associated with the overexpression of multiple members of the EGF family including TGF-α, heparin-binding EGF-like growth factor (HB-EGF), amphiregulin, and betacellulin, these findings raise the possibility that loss of TIP30 in PDAC may lead to EGFR up-regulation in the face of high levels of EGFR ligands which would otherwise promote EGFR degradation. Moreover, loss of TIP30 enhances susceptibility to tumorigenesis and has been associated with metastatic breast cancer. Taken together, these observations show that increased miR-10b in PDAC may promote metastasis and disease aggressiveness by suppressing TIP30 and upregulating EGFR.

miR-10b has been reported to promote invasion in several cancer types by suppressing the expression of various suppressors of metastasis. In breast cancer, in addition to targeting HOXD10, miR-10b enhances invasion by targeting syndecan-1 (SDC-1) which is known to suppress metastases. Moreover, miR-10b may act by suppressing KLF4, a zinc finger transcription factor which can also suppress metastasis formation, and NF-1 which attenuates ras activity. Although in the present disclosure miR-10b did not alter HOXD10 expression, it decreased the expression of SDC1, NF1, and KLF4. It is possible that additional factors bind to the non-targeted mRNA moieties in PCCs, rendering their sites inaccessible to miR-10b, as has been reported for miR-34a.

Gene profiling identified two new potential targets downregulated by miR-10b, RAP2A and EPHB2. Bioinformatic analysis predicted that both genes harbor at least one miR-10b binding site, showing that they are direct targets of miR-10b. RAP2A has been implicated in cell adhesion and is induced by LKB1, whose loss of function is associated with altered cell polarity and enhanced metastases in lung cancer. Similarly, loss of EPHB2 has been associated with increased metastasis in colorectal cancer. This disclosure also found that miR-10b enhanced EGF and TGF-induced expression of EMT-associated genes decreasing E-cadherin levels while increasing N-cadherin, vimentin, Twist, Snail and Zeb1 levels. Taken together, these findings show that miR-10b modulates a repertoire of genes in PCCs whose downregulation enhances the metastatic process while promoting EMT, which further increases the motility and metastatic potential of these cells.

A number of reports have pointed to reciprocal modulatory interactions between specific miRs and members of the EGF receptor family. Thus, EGFR activation leads to increased expression of miR-21, miR-221 and miR-222. Conversely, miR-7 and miR-133b suppress EGFR expression, whereas miR-125a and 125b suppress HER2/HER3, and miR-205 targets HER3 but not other HERs. This appears to be the first disclosure showing the potential connection between miR-10b and EGFR signaling. These findings also demonstrate that miR-10b facilitates potentially deleterious interactions between EGFR and TGF-β pathways. Other information regarding the diagnosis of pancreatic cancer by analyzing bodily fluids can be found in U.S. provisional patent application No. 61/833,571 filed on Jun. 11, 2013, and incorporated herein by reference in its entirety.

### Patient Cohort

Selected Patients from a cohort who had been prospectively enrolled between July, 2012 and February, 2014 into a Pancreatobiliary Diseases Database and Biological Repository at Indiana University School of Medicine. This database includes individuals with PDAC, CP, or other benign pancreatobiliary diseases undergoing endoscopic ultrasound (EUS) or endoscopic retrograde cholangiopancreatography (ERCP) at Indiana University Health University Hospital, Indianapolis, Ind. This represents a tertiary referral center for individuals with advanced pancreatic disease such as CP and PDAC. From this Biological Repository, patients having available plasma, bile, pancreatic juice, or some combination were selected. All samples were procured immediately prior to or during their endoscopy.

All patients in the Repository with a confirmed tissue diagnosis of PDAC and no prior therapy for PDAC are thought to have been included. To identify miRNA having the highest accuracy in distinguishing PDAC from CP and controls, a training cohort derived from the Repository was established. This included an arbitrary subgroup of the individuals with PDAC and an age-matched sample of individuals enrolled into the Repository having a confirmation of CP or other benign biliary disorders (controls) with available specimens for analysis. All patients with CP were undergoing ERCP for the treatment of pathology (i.e., strictures) of the bile, pancreatic, or both ducts. All patients classified as controls were undergoing ERCP for the treatment of choledocholithiasis. To test the performance characteristics of a miRNA panel developed from the training cohort, a validation cohort comprised of individuals having the same diagnoses using a separate, random sample derived from the Repository was established.

Relevant clinical data were collected at the time of the procedure, and a diagnosis of PDAC required cytopathological confirmation. All individuals with PDAC were enrolled prior to the initiation of therapy (treatment-naïve). Individuals with CP were classified by the Cambridge criteria on computed tomography, ERCP, or both.[18] Control subjects had previously undergone normal cross sectional imaging of the pancreas. Prior to subject enrollment, the local responsible Institutional Review Board approved this study protocol and each subject signed informed consent.

### Sample Procurement

After signing informed consent and before the onset of endoscopy, no more than 20 mL of blood was collected and equally distributed into EDTA-coated tubes. Among subjects undergoing ERCP, 1-5 mL of bile and/or pancreatic juice was aspirated and collected in an uncoated tube. Specimens were initially stored at 4-8° C., and then rapidly processed by centrifugation followed by collection of supernatant. After processing, all supernatants were stored at −80° C. until analysis.

miRNA Selection and Assay Methodology

Investigators analyzing samples (AG, SM, MK) were blinded to the underlying diagnosis. Plasma, pancreatic juice and bile aspirates for 10 miRNA candidates (-10b, -21, -30c, -106b, -132, -155, -181a, 181b, -196a, and -212) having a known or suspected association with PDAC were assayed. miR-10b, -21, -196a, and -155 are overexpressed in PDAC. [19, 20] Compared to normal or CP patients, levels of miRNA-10b levels were elevated in archival plasma samples from individuals with PDAC.[12] Importantly, miRNA-10b is one of the most frequently up-regulated miRNAs in PDAC, and biopsies from endoscopic ultrasound-derived fine needle aspirates (EUS-FNA) were used to correlate decreased miRNA-10b expression in the cancer cells in PDAC with improved survival, response to neoadjuvant radio-chemotherapy, and delayed time to metastasis.[19] Similarly, miR-21 is overexpressed in PDAC and has been proposed as a therapeutic target in this cancer,[21] whereas miR-132 and miR-212 are expressed at high levels in PDAC and target RB, [22] thereby contributing to RB dysfunction which has an important role in PDAC pathobiology.[23] Moreover, miR-106b targets both RB [24] and p21, [25] and loss of p21 is observed in cases of RB dysfunction in PDAC. [23]

Although the exact functions of miR-30c, miR-181a and -181b in PDAC are not known, miR-30c is an oncomir that is upregulated by the EGF receptor, and this receptor has a crucial role in PDAC[26, 27], whereas miR-181a and -181b are known to be overexpressed in PDAC. [28, 29] These 10 plasma miRNAs represent a broad variety of functions in PDAC and have high potential for release into the circulation. It is also of note that many patients with PDAC and CP present with bile or pancreatic duct obstruction. This usually prompts ERCP for drainage and intraductal tissue sampling in conjunction with EUS-FNA. Tissue sampling is operator dependent and cytopathology inaccurate in the setting of CP, so a diagnostic test requiring aspiration of bile or pancreatic juice would be clinically useful in certain cases.

Total RNA was isolated from plasma, bile, and pancreatic juice samples using Trizol-LS® (Life Technologies, Carlsbad, Calif., USA). cDNA was generated using 10 ng of RNA in conjunction with miR-10b, -21, -30c, -106b, -132, -155, -181a, -181b, -196a, -212, or -425-5p RT primers and a miRNA reverse transcription kit (Life Technologies) according to the manufacturer's recommendations. Quantitative PCR (qPCR) was performed for each miRNA using Taqman® miRNA expression assay reagents. As an internal control, expression levels for all candidate miRNAs were normalized to miR-425-5p, which was expressed at similar levels in all samples, exhibiting <1 cycle threshold (Ct) difference across the samples.[30] miR-16, -23a, and -93 were evaluated as endogenous controls, but these miRNAs exhibited greater inter-sample variability than miR-425-5p. After normalization to miR-425-5p (ΔCt), the ΔCt values for miRNAs in controls were averaged and subtracted from the ΔCt values of each individual sample (ΔΔCt) and expression levels were calculated using the 2-^{ΔΔCt} method, which indicates a two-fold difference per every difference in normalized Ct values.[31]

### Statistical Analysis

Sample characteristics, laboratory characteristics, and miRNA expression levels of patients in the PDAC, CP and control groups were compared using ANOVA for normally distributed continuous variables, non-parametric Kruskal-Wallis tests for non-normally distributed continuous variables, and Pearson Chi-square tests for categorical variables. Descriptive statistics were reported using mean and standard deviation for normally distributed, continuous variables and median and interquartile range (IQR) for non-normally distributed continuous variables. For each variable, pairwise comparison was performed with p-values adjusted using the Bonferroni approach. [32]

A step in the development of the PDAC Signature Panel involved bivariable analyses using logistic regression in the training cohort in order to determine the relationship between expression level of each miRNA and presence/absence of PDAC (PDAC vs. CP or control). Predictive performance of each miRNA was described using the Receiver Operating Curve (ROC) and the area under the curve (AUC), with excellent accuracy defined as AUC>0.90. The predictive performance of each miRNA in discriminating PDAC vs. CP, PDAC vs. control and CP vs. control was evaluated. In order to explore dichotomization of the expression level, miRNAs having excellent accuracy in univariate analysis of PDAC prediction were further examined using a classification tree model.

Multivariable analyses were then performed using logistic regression in which dichotomized expression levels of all miRNAs with excellent accuracy were included in a forward stepwise selection procedure (p<0.20 for entering and staying in the model) to determine the final predictors for the PDAC Signature Panel. Due to the exploratory nature of the analyses, p<0.20 was chosen in the multivariable model. Finally, a point scoring system in which points were assigned to miRNAs using coefficients from the final logistic regression model was constructed. Points associated with each miRNA by dividing the corresponding coefficient by the lowest coefficient in the final model and rounding to the nearest integer were calculated. The Panel score for each patient was computed by adding the points for all miRNAs in the final model. The PDAC Signature Panel scoring system was repeated using plasma alone and bile alone.

In order to validate the PDAC Signature Panel, the point scoring system constructed in the training cohort was applied to the validation cohort by determining the risk points for each patient in the validation cohort. Performance of these Signature Panels was evaluated using sensitivity and specificity.

Since there were limited pancreatic juice samples from patients, the differential expression of miRNA was described using data derived from both training and validation cohorts for patients with PDAC and CP in the secondary analysis. Additionally, for descriptive purposes, the differences in miRNA expression among PDAC individuals having metastatic (stage IV) and non-metastatic (stage I-III) disease was measured. Statistical analysis was performed using SAS v. 9.3 (Cary, N.C.) and R Project software (Vienna, Austria).

### Study Population

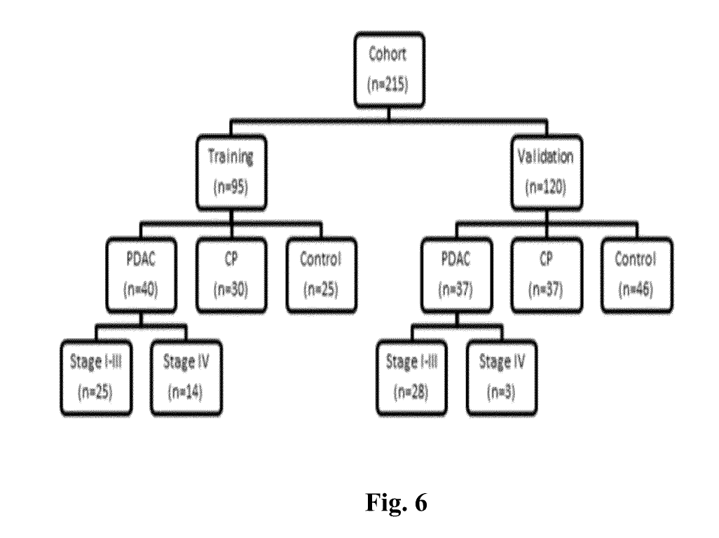

The study included a total of 215 individuals identified in the Biological Repository, with 95 subjects comprising the training and 120 comprising the validation cohort (FIG. 6). Of individuals with PDAC, 53/77 (69%) were staged T1-T3 at the time of specimen procurement. Clinical characteristics are summarized in Table 1. There were no significant differences in age and sex in the training cohort; in the validation cohort, subjects with PDAC were significantly older. Tobacco use was less prevalent among control subjects in the training cohort, but similar between PDAC and CP individuals. Alcohol use was less prevalent among control subjects in the validation cohort, but similar between PDAC and CP individuals in both the training and validation cohorts.

TABLE 1
Patient characteristics.

| | | | | | | | |
|---|---|---|---|---|---|---|---|
| | | | | | | PDAC | |
| | | | | P | PDAC | vs. | CP vs. |
| Variable | PDAC | CP | Control | value | vs. CP | Control | Control |

Training Cohort

| | N = 40 | | N = 30 | | N = 25 | | | | | |
|---|---|---|---|---|---|---|---|---|---|---|
| Age, mean (SD)* | 66.5 | (9.8) | 64.0 | (10.4) | 66.4 | (10.8) | 0.567 | | | |
| Female Sex, n (%) | 24 | (60%) | 15 | (50%) | 15 | (60%) | 0.658 | | | |
| Tobacco use, status, n (%) | | | | | | | 0.012 | 0.083 | 1.000 | 0.016 |
| Never | 18 | (47.4%) | 6 | (21.4%) | 13 | (61.9%) | | | | |
| Ever | 13 | (34.2%) | 9 | (32.1%) | 6 | (28.6%) | | | | |
| Current | 7 | (18.4%) | 13 | (46.4%) | 2 | (9.5%) | | | | |
| Alcohol use, status | | | | | | | 0.084 | | | |
| Never | 20 | (51.3%) | 15 | (55.6%) | 17 | (81%) | | | | |
| Ever | 13 | (33.3%) | 11 | (40.7%) | 3 | (14.3%) | | | | |
| Current | 6 | (15.4%) | 1 | (3.7%) | 1 | (4.8%) | | | | |
| Tobacco and alcohol use, | 13 | (35.1%) | 12 | (44.4%) | 2 | (9.5%) | 0.030 | 1.000 | 0.097 | 0.025 |
| n (%) | | | | | | | | | | |

Validation cohort

| | N = 37 | | N = 37 | | N = 46 | | | | | |
|---|---|---|---|---|---|---|---|---|---|---|
| Age, mean (SD) | 65.2 | (11.9) | 47.7 | (12.2) | 48.8 | (15.8) | <0.001 | <0.001 | <0.001 | 1.000 |
| Female Sex, n (%) | 18 | (48.7%) | 19 | (51.4%) | 31 | (67.4%) | 0.170 | | | |
| Tobacco use, status, n (%) | | | | | | | 0.274 | | | |
| Never | 13 | (35.1%) | 8 | (21.6%) | 15 | (33.3%) | | | | |
| Ever | 13 | (35.1%) | 9 | (24.3%) | 14 | (31.1%) | | | | |
| Current | 11 | (29.8%) | 20 | (54.1%) | 16 | (35.6%) | | | | |
| Alcohol use, status | | | | | | | 0.030 | 0.455 | 1.000 | 0.018 |
| Never | 22 | (61.1%) | 15 | (40.5%) | 33 | (73.3%) | | | | |
| Ever | 9 | (25%) | 17 | (46%) | 7 | (15.6%) | | | | |
| Current | 5 | (13.9%) | 5 | (13.5%) | 5 | (11.1%) | | | | |
| Tobacco and alcohol use, | 11 | (30.6%) | 21 | (56.8%) | 10 | (22.2%) | 0.004 | 0.072 | 1.000 | 0.004 |
| n (%) | | | | | | | | | | |

PDAC = pancreatic ductal adenocarcinoma;
CP = chronic pancreatitis;
SD = standard deviation
*To minimize the potential of age being a confounder in miRNA expression between groups, subjects were age-matched in the training cohort.
Notes:
Continuous variables were compared that are normally distributed using ANOVA (age), likelihood ratio chi-square test for categorical variables having low expected cell counts, and other variables using the Pearson Chi-square test. Pairwise comparisons were performed using the Bonferroni multiple comparison approach.

Laboratory characteristics at the time of specimen procurement are summarized in Table 2. Comparative statistics for carbohydrate antigen 19-9 (CA19-9) were not performed because values were only available for individuals with PDAC. Compared to individuals with CP and controls, median serum bilirubin and the proportion of cases having at least mild elevation in serum bilirubin (defined as >34.2 μmol/L) were significantly greater in PDAC cases. However, the inclusion of serum bilirubin as a potential confounding variable did not impact miRNA expression or the performance of our diagnostic panels detailed below. Serum albumin and total protein were significantly lower in subjects with PDAC in the validation cohort, but similar in the training cohort; observed differences were not clinically relevant (differences<1.0 for each measure). White blood count and calcium levels were similar across groups in both cohorts.

TABLE 2
Laboratory characteristics.

| | | | | | | | |
|---|---|---|---|---|---|---|---|
| | | | | P | PDAC | PDAC vs. | CP vs. |
| Variable | PDAC | CP | Control | value | vs. CP | Control | Control |

Training Cohort

| | N = 40 | | N = 30 | | N = 25 | | | | | |
|---|---|---|---|---|---|---|---|---|---|---|
| CA 19-9, median (IQR) | 385 | (1689) | | | | | | | | |
| Total bilirubin, | 90.6 | (191.5) | 10.3 | (3.4) | 12.0 | (18.8) | <0.001 | <0.001 | 0.001 | 1.000 |
| μmol/L (median, IQR) | | | | | | | | | | |
| At least mild elevation | 20 | (55.6%) | 1 | (3.5%) | 4 | (20.0%) | <0.001 | <0.001 | 0.024 | 0.176 |
| (bilirubin >34.2 μmol/L), | | | | | | | | | | |
| n (%) | | | | | | | | | | |
| WBC, k/mcL | 7.5 | (3.1) | 7.8 | (2.6) | 5.9 | (2.5) | 0.107 | | | |
| (median, IQR) | | | | | | | | | | |
| Albumin, μmol/L | 5.4 | (0.7) | 5.7 | (0.9) | 5.7 | (0.6) | 0.137 | | | |
| (mean, std) | | | | | | | | | | |
| Total protein, | 9.9 | (1.2) | 10.4 | (0.9) | 10.0 | (0.7) | 0.078 | | | |
| μmol/L (mean, std) | | | | | | | | | | |
| Calcium, mmol/L | 2.3 | (0.2) | 2.4 | (0.1) | 2.3 | (0.2) | 0.674 | | | |
| (mean, std) | | | | | | | | | | |

Validation cohort

| | N = 37 | | N = 37 | | N = 46 | | | | | |
|---|---|---|---|---|---|---|---|---|---|---|
| CA 19-9, median | 742.5 | (1477) | | | | | | | | |
| (IQR) | | | | | | | | | | |
| Total bilirubin, | 198.4 | (222.3) | 6.8 | (5.1) | 15.4 | (20.5) | <0.001 | <0.001 | <0.001 | <0.001 |
| μmol/L (median, IQR) | | | | | | | | | | |
| At least mild | 26 | (78.8%) | 2 | (5.4%) | 9 | (20.5%) | <0.001 | <0.001 | <0.001 | 0.120 |
| elevation | | | | | | | | | | |
| (bilirubin >34.2 μmol/L), | | | | | | | | | | |
| n (%) | | | | | | | | | | |
| WBC, k/mcL | 8.0 | (4.6) | 7.6 | (1.6) | 7.7 | (3) | 0.859 | | | |
| (median, IQR) | | | | | | | | | | |
| Albumin, μmol/L | 5.1 | (1.2) | 5.7 | (0.7) | 5.7 | (0.7) | 0.005 | 0.039 | 0.005 | 1.000 |
| (mean, std) | | | | | | | | | | |
| Total protein, | 9.4 | (1.3) | 10.0 | (0.7) | 10.4 | (1.2) | 0.001 | 0.074 | <0.001 | 0.285 |
| μmol/L (mean, std) | | | | | | | | | | |
| Calcium, mmol/L | 2.3 | (0.1) | 2.3 | (0.1) | 2.4 | (0.2) | 0.070 | | | |
| (mean, std) | | | | | | | | | | |

CA19-9 = carbohydrate antigen 19-9;
WBC = white blood count;
IQR = interquartile range
Notes:
Continuous variables that are normally distributed are compared using ANOVA (Albumin, total protein, calcium) and others are compared using the non-parametric Kruskal-Wallis test (total bilirubin, WBC). Likelihood ratio chi-square test was used for the categorical variable mild bilirubin elevation due to low cell counts. Pairwise comparisons were performed using the Bonferroni multiple comparison approach.

miRNA Expression

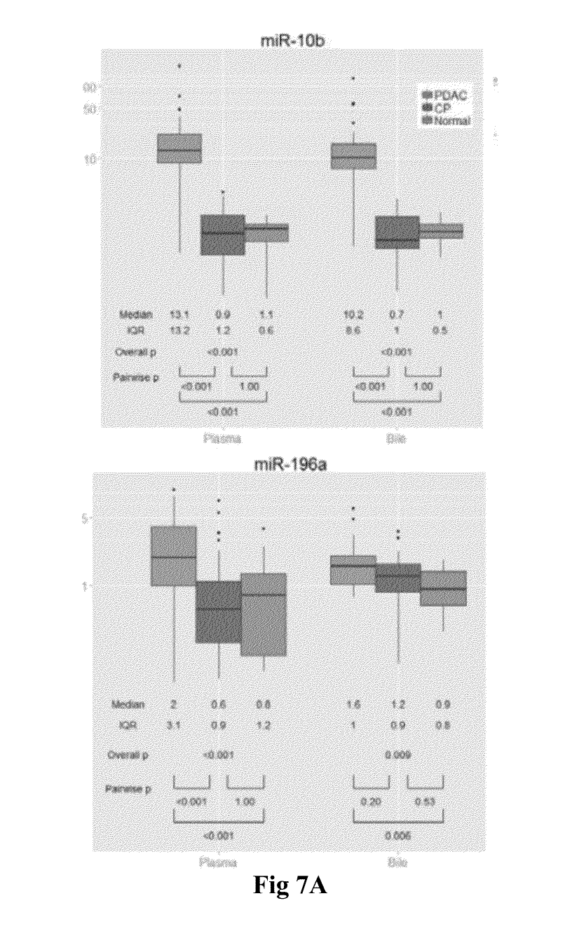

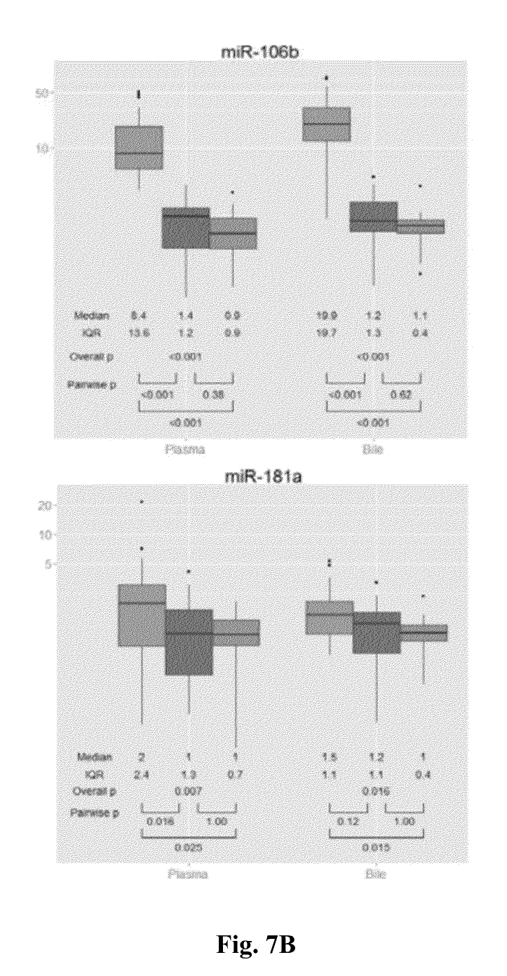

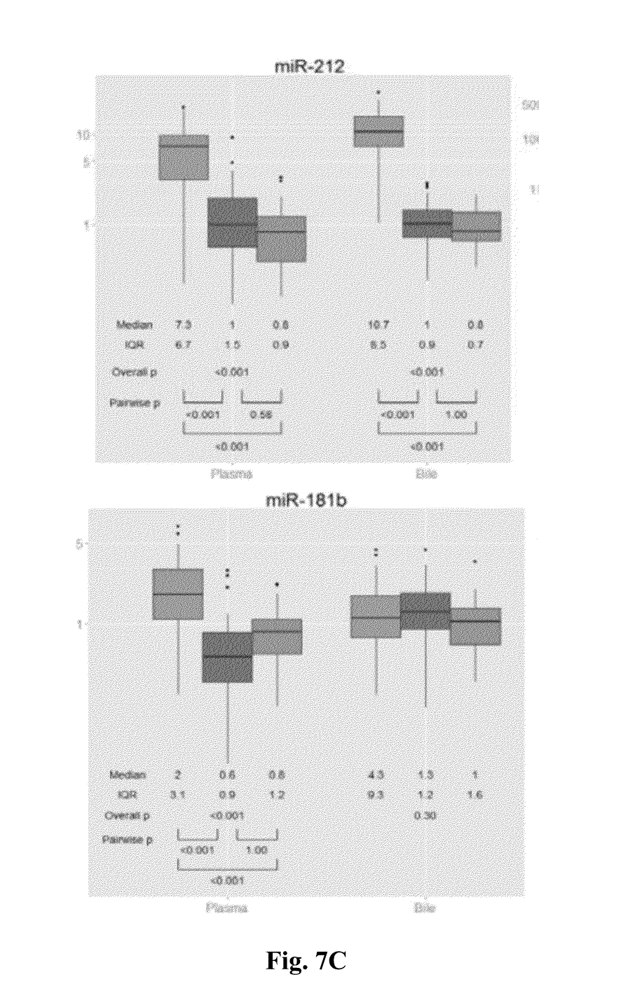

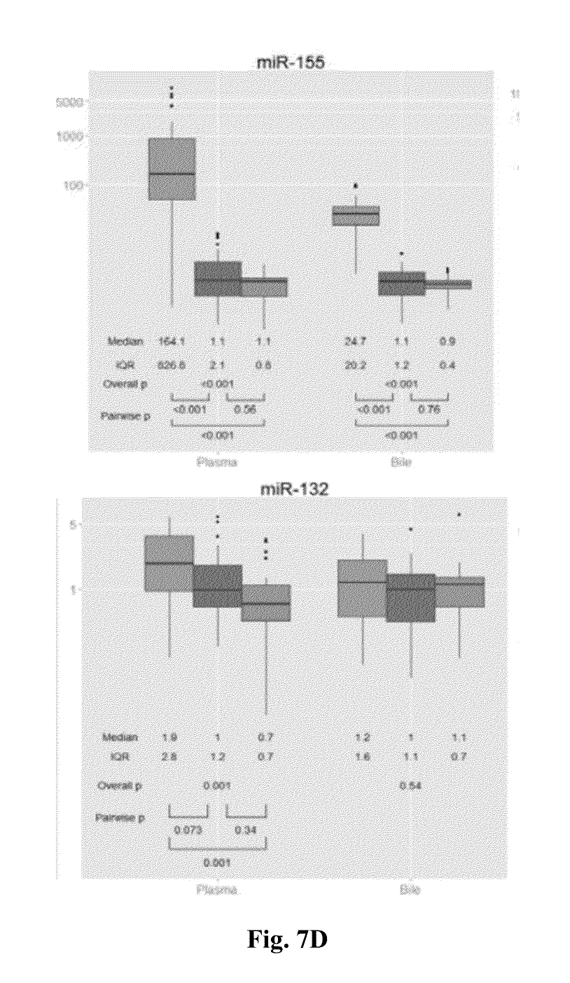

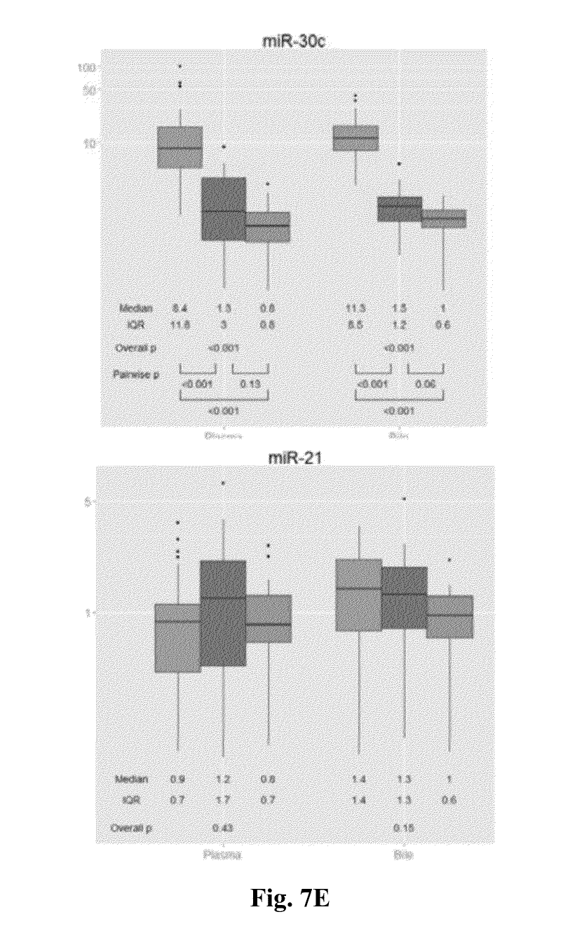

Ten miRNA in plasma and bile in the training cohort were evaluated: plasma was available for analysis in all but 1 control and bile in all but 15 PDAC, 7 CP, and 3 controls (FIG. 7, supplementary table 1). Of the plasma miRNA, only miR-21 was similar across groups (p=0.426). miR-10b, -30c, -106b, -155, -181b, -196a, and -212 were significantly different across all three groups with a p value<0.001 and between individuals with PDAC and CP (p<0.001). miR-132 and -181a were also significantly different across all three groups (p=0.001 and 0.007 respectively), with statistically significant differences persisting in pairwise comparison of PDAC and CP for only -181a (p=0.016). In bile, all miRNA were significantly different across groups except for miR-21 (p=0.151), -132 (p=0.535) and -181b (p=0.297). Differences in miR expression in bile persisted in pairwise comparisons of PDAC vs. CP except for miR-181a (p=0.115) and -196a (p=0.198); in pairwise comparisons of PDAC vs. control, differences in bile miR expression persisted.

Compared to PDAC individuals with stage I-III disease (n=53), those having stage IV disease at the time of specimen procurement (n=17, with 7 individuals lacking stage data) had similar plasma miRNA profiles for each of the 10 miRNA measured. Although not meeting statistical significance, median (IQR) expression of miR-132 (n=24) was higher among subjects with stage IV disease (2.01 (1.44) vs. 0.57 (1.58), p=0.0926); miR-155, and -181b expression in bile (n=24) were lower among subjects with stage IV disease (miR-155: 14.59 (18.29) vs. 24.7 (20.9), p=0.0792; miR-181b: 0.62 (0.84) vs. 1.22 (0.82), p=0.0448). All other bile miRNA were expressed similarly between those with stage IV and I-III disease.

### PDAC Signature Panel

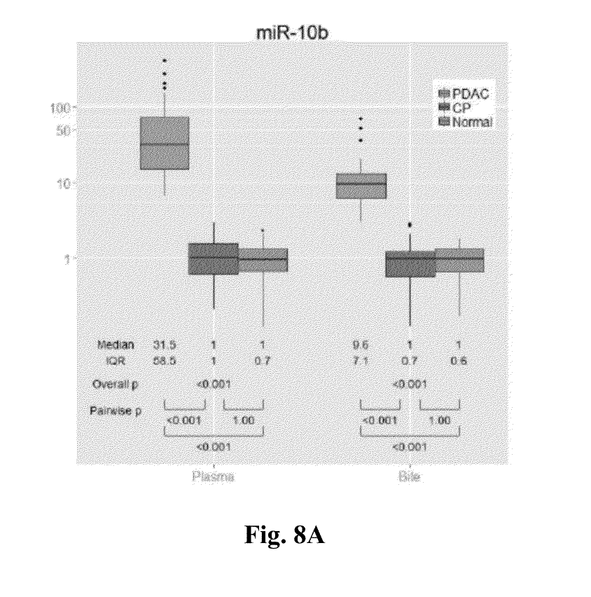

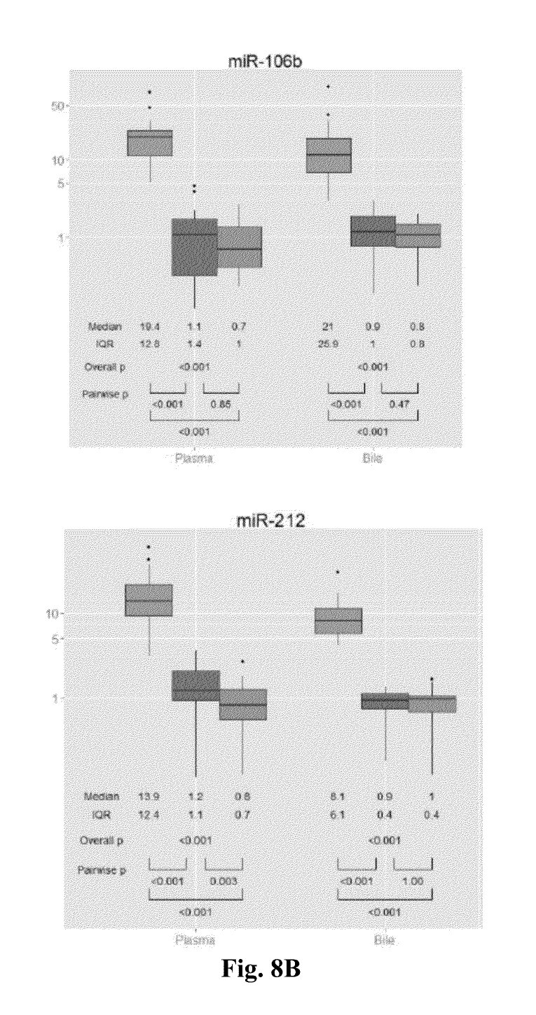

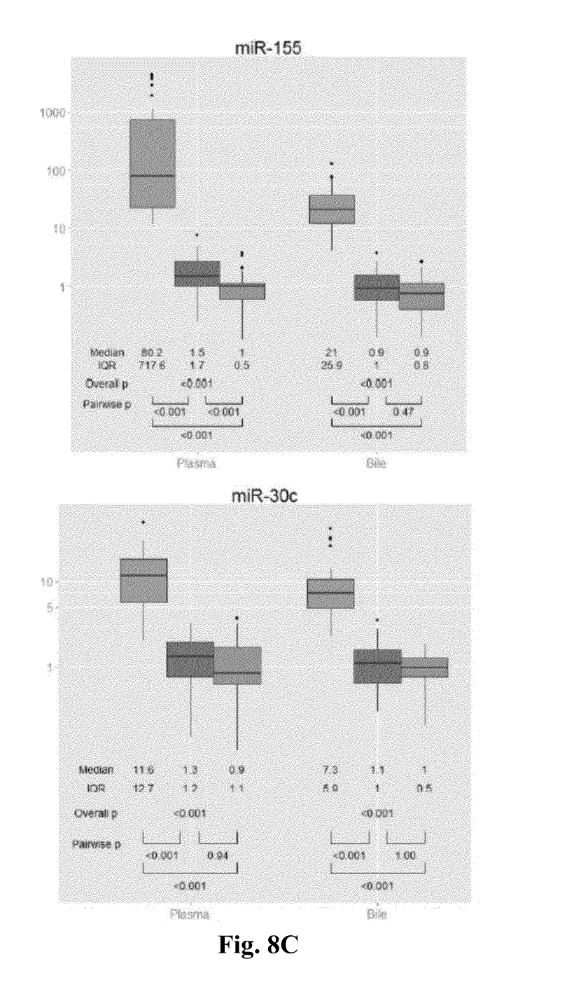

Bivariable logistic regression analyses showed that 5 miRNAs (10b, 30c, 106b, 155, and -212) in plasma and bile provided excellent accuracy (AUC>0.90) for distinguishing PDAC patients from others (CP+control) based on the training cohort (Table 5). These miRNAs also provided excellent accuracy for distinguishing PDAC from controls. In addition, each miRNA had good (plasma miR-212, defined as AUC>0.80) or excellent (all other plasma miR and all five bile miR) accuracy in distinguishing PDAC from CP subjects. Therefore, these miRNAs to build a PDAC Signature Panel and apply to the validation cohort were selected. Based on classification tree analyses, the thresholds for dichotomizing the expression levels were 3.579, 4.873, 2.920, 10.680, and 2.013 for plasma miR-10b, -30c, -106b, -155 and -212, respectively. For the same five miRNA derived from bile, thresholds were 3.497, 3.933, 5.261, 5.232 and 4.163, respectively. Considering these thresholds, the sensitivity and specificity of these individual miRNA derived from plasma and bile are computed and summarized in Table 3. Assays of plasma and bile for miR-10b, -155, -106b, -30c and -212 using samples derived from the validation cohort, performed similarly to the training cohort (FIG. 8).

TABLE 3
Performance characteristics of plasma and bile miRNA for diagnosing
PDAC in the training cohort (n = 95).

| | | | | | | |
|---|---|---|---|---|---|---|
| | True | | | | | |
| | Pos- | True | False | False | | |
| Candidate | itives | Negatives | Positives | Negatives | Sen- | Spe- |
| miRNA | (n) | (n) | (n) | (n) | sitivity | cificity |

Plasma

| | | | | | | |
|---|---|---|---|---|---|---|
| miR-10b | 38 | 54 | 0 | 2 | 95% | 100% |
| miR-30c | 29 | 52 | 2 | 11 | 73% | 96% |
| miR-106b | 40 | 53 | 1 | 0 | 100% | 98% |
| miR-155 | 37 | 54 | 0 | 3 | 93% | 100% |
| miR-212 | 36 | 45 | 9 | 4 | 90% | 83% |

Bile

| | | | | | | |
|---|---|---|---|---|---|---|
| miR-10b | 24 | 45 | 0 | 1 | 96% | 100% |
| miR-30c | 24 | 44 | 1 | 1 | 96% | 98% |
| miR-106b | 24 | 45 | 0 | 1 | 96% | 100% |
| miR-155 | 24 | 45 | 0 | 1 | 96% | 100% |
| miR-212 | 24 | 45 | 0 | 1 | 96% | 100% |

Using the dichotomized differential miRNA expression level in the training cohort, PDAC Signature Panels using plasma miRNA alone and bile miRNA alone (Table 4) were constructed. Using plasma miRNA alone, forward stepwise selection procedure selected miR-10b and -106b, with the other three miR dropping from the model for p value>0.20 due to high correlation between each of the miR. Based on coefficients derived from the final logistic regression model including plasma miR-10b (parameter estimate±SE 3.83±1.87, p=0.0405) and -106b (5.18±1.87, p=0.0055), one point was assigned for high miR-10b (>3.579) and one point for high miR-106b (>2.920). Using a plasma Panel score≧2 to diagnose PDAC, 95% sensitivity (95% CI, 83%-99%) and 100% specificity (95% CI, 93%-100%) was observed in the training cohort and 29/29=100% sensitivity (95% CI, 88%-100%) and 75/75=100% specificity (95% CI, 95%-100%) was observed in the validation cohort.

TABLE 4
Performance Characteristics of PDAC Signature Panels derived from plasma or bile

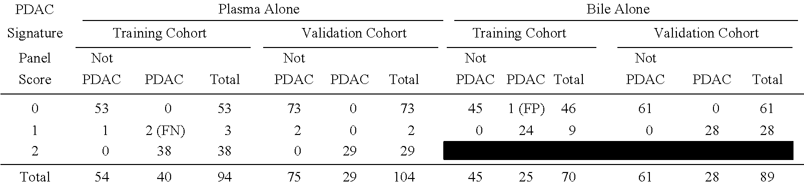

FP = false positive; FN = false negative; PDAC = pancreatic ductal adenocarcinoma
Shaded boxes depict values equal to or above threshold PDAC Signature Panel scores derived from regression models using plasma miR alone (≧2) and
bile miR alone (≧1).

TABLE 5
Performance characteristics of miRNA for distinguishing PDAC from CP
and control subjects (training cohort, n = 95)

| | | | | | |
|---|---|---|---|---|---|
| Candidate | PDAC vs. | PDAC vs. | CP vs. | PDAC vs. | |
| miRNA | CP | Control | Control | Other | Threshold |

Plasma

| | | | | | |
|---|---|---|---|---|---|
| miR-10b | 0.982 | 1.000 | 0.494 | 0.980 | 3.579 |
| miR-21 | 0.589 | 0.485 | 0.572 | 0.556 | |
| miR-30c | 0.902 | 0.984 | 0.625 | 0.938 | 4.873 |
| miR-106b | 0.998 | 1.000 | 0.609 | 0.999 | 2.92 |
| miR-132 | 0.659 | 0.757 | 0.634 | 0.703 | |
| miR-155 | 0.971 | 0.979 | 0.601 | 0.975 | 10.68 |
| miR-181a | 0.687 | 0.698 | 0.495 | 0.692 | |
| miR-181b | 0.873 | 0.786 | 0.680 | 0.834 | |
| miR-196a | 0.763 | 0.773 | 0.466 | 0.767 | |
| miR-212 | 0.878 | 0.927 | 0.596 | 0.900 | 2.013 |

Bile

| | | | | | |
|---|---|---|---|---|---|
| miR-10b | 0.974 | 0.965 | 0.458 | 0.970 | 3.497 |
| miR-21 | 0.485 | 0.645 | 0.646 | 0.578 | |
| miR-30c | 0.991 | 1.000 | 0.678 | 0.996 | 3.933 |
| miR-106b | 0.983 | 0.991 | 0.597 | 0.987 | 5.261 |
| miR-132 | 0.590 | 0.562 | 0.538 | 0.576 | |
| miR-155 | 0.986 | 0.995 | 0.586 | 0.990 | 5.232 |
| miR-181a | 0.671 | 0.733 | 0.569 | 0.701 | |
| miR-181b | 0.550 | 0.580 | 0.636 | 0.513 | |
| miR-196a | 0.637 | 0.772 | 0.599 | 0.703 | |
| miR-212 | 0.981 | 0.982 | 0.538 | 0.981 | 4.163 |

AUC = Area under the curve; Threshold denotes the miRNA expression level corresponding to the AUC for PDAC vs. All other (CP + controls).
For reference, accuracy is graded from fail to excellent based on the following AUC thresholds: Excellent ≧ 0.90; Good ≧ 0.80; Fair ≧ 0.70; Poor ≧ 0.60; Fail < 0.60.

TABLE 6
Relative expression of miRNA in plasma and bile among individuals with PDAC, CP,
and controls

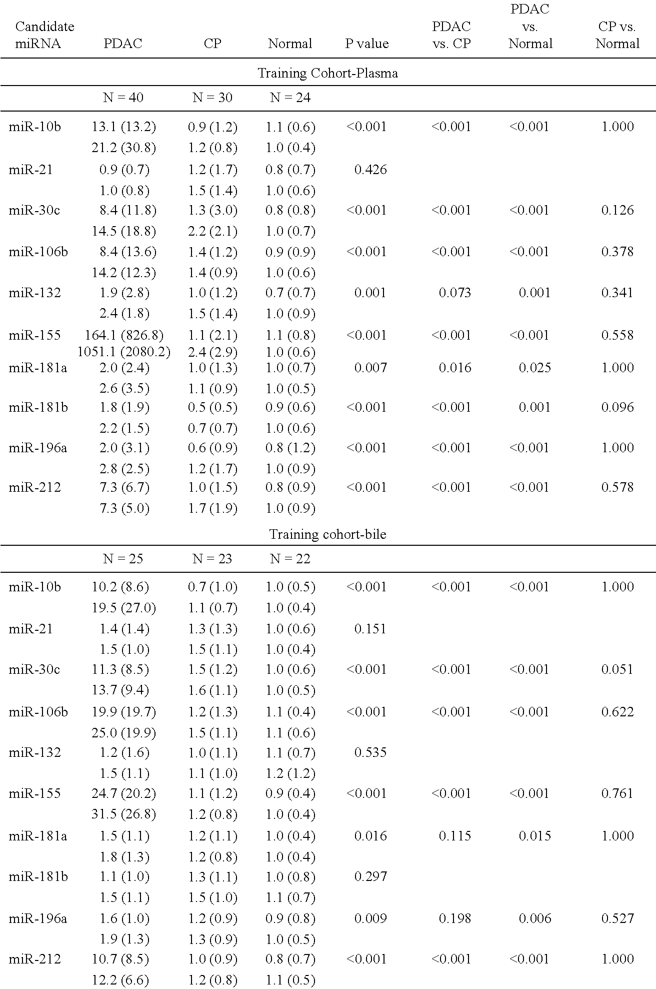

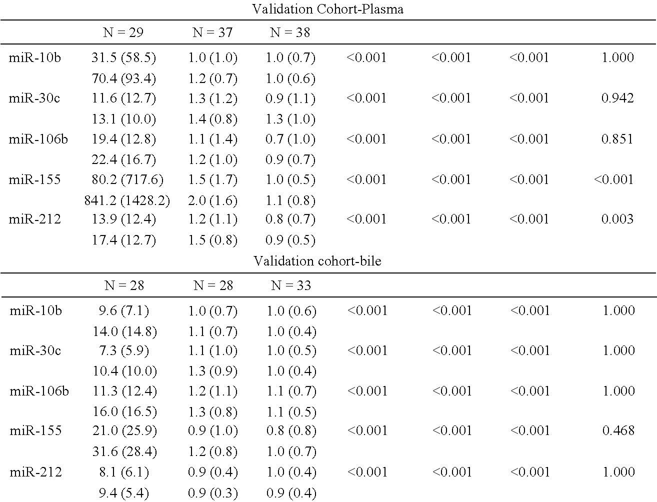

All miRNA levels are nonparametric and hence median (interquartile range) is reported. We also include mean (standard
deviation) in gray font for descriptive purposes. P-values are obtained based using the Kruskal-Wallis test and pairwise
p-values are adjusted using the Bonferroni multiple comparison approach.

A similar model was constructed using bile miRNA alone. The forward stepwise selection procedure confirmed that each of the five miRNA meeting the predefined threshold level performed identically in distinguishing PDAC from other etiologies. The addition of two or more bile miRNA did not improve the performance of the Panel. Therefore, one point was assigned to a patient if any of the following miRNA exceeded their threshold score: miR-10b (>3.497), -106b (5.261), -155 (5.232), -212 (>4.163). Using a threshold bile Panel score≧1, 24/25=96% sensitivity (95% CI, 80%-100%) and 45/45=100% specificity (95% CI, 92%-100%) in the training cohort and 28/28=100% sensitivity (95% CI, 88%-100%) and 61/61=100% specificity (95% CI, 94%-100%) was observed in the validation cohort (Table 4).

The inclusion of serum bilirubin in our regression models (that is, controlling for differences in serum bilirubin) did not impact the performance characteristics of our PDAC Panels using plasma (p=0.7269) or bile miRNA (p=0.9154).

### Pancreatic Juice

Since pancreatic juice was available in a limited number of individuals with PDAC (n=9) and CP (n=34) in the entire cohort, the differential expression of miR-10b, -155, -106b, -30c, and -212 between these groups are presented. On univariate analysis, the AUC=1.0 (100% accuracy in distinguishing PDAC from CP) for all miRNA except miR-30c (AUC=0.941). Thresholds based on the classification tree were 4.42 for miR-155, 4.54 for -106b, 3.41 for -10b, 3.69 for -212, and 3.27 for -30c. Similar to models for plasma alone and bile alone, a PDAC Panel score≧1 correctly diagnosed individuals with PDAC from those with CP.

### Discussion

In their lifetime, PDAC will develop in 1 of 68 Americans, and only 6% of affected individuals will survive five years.[33] Clinical trials touting the incremental benefit of chemotherapeutic agents such as gemcitabine, combination oxaliplatin/leucovorin/irinotecan/fluorouracil (FOLFIRINOX)[34], and albumin-bound paclitaxel[35] report survival benefits that are quantified in months. Given these dismal statistics, there is substantial interest in developing novel tests to identify PDAC at an earlier stage or even in precursor lesions such as pancreatic intraepithelial neoplasia (PanIN), or early-stage intraductal papillary mucinous neoplasm (IPMN).[36, 37, 38] The need for superior diagnostics applies to patients with or without CP who present with indeterminate bile or pancreatic duct strictures, where ERCP-based tissue sampling techniques have limited sensitivity and where bile or pancreatic juice aspirates may apply. Case control studies evaluating different miRNA profiles in whole blood[39, 40] or plasma/serum[12, 28, 41, 42] have yielded varying results using different miRNA signals. Given the high accuracy of individual miRNA and the PDAC Signature Panel derived from our cohort, plasma appears to be a superior medium than serum[41] or whole blood[40, 43] for this indication.

In the present analysis, the performance of the panels that incorporated the differential expression of miRNA each having excellent accuracy in distinguishing PDAC from CP—an at-risk population where current tissue sampling techniques have lower sensitivity—and controls is superior to historical populations using CA19-9.[44] A limitation of CA19-9 is its diminished specificity in the setting of obstructive jaundice; the miRNA studied were unaffected by the presence of jaundice, and inclusion of serum bilirubin in our panels did not significantly impact the results. The reference populations (CP patients having pancreatobiliary duct pathology and controls having choledocholithiasis) reflect a “real-world” cohort of individuals with a variety of pancreatobiliary diseases, some of which (e.g., CP) may mimic PDAC and in whom a peripheral biomarker would be most useful. A control population with benign disorders as opposed to a purely healthy control population in an effort to minimize selection bias was deliberately chosen. For cancer screening, a PDAC Signature Panel would unlikely apply to the general population but rather high-risk populations such as those with a family history and CP.

miRNA Stability in Plasma, Bile, and Pancreatic Juice

Plasma miRNAs are stable over a wide pH range and do not degrade when plasma is subjected to multiple freeze-thaw cycles or to boiling.[45, 46] This remarkable stability has been attributed to their binding to Argonaute2 (Ago2), an RNA-binding protein, as well as to high density lipoproteins.[47] Therefore, reproducible results of miRNA expression in plasma, bile, and pancreatic juice was generated. Importantly, in clinical practice, patients with bile duct and pancreatic duct strictures are a diagnostic conundrum. ERCP- and EUS-based tissue sampling techniques have reduced sensitivity for distinguishing PDAC from CP and other benign etiologies of stricture, so aspiration of bile, pancreatic juice, or both is an attractive alternative to cholangiopancreatoscopy and fluorescence in situ hybridization—current “second-tier” diagnostic tests used in clinical practice.[48] Additionally, tissue sampling from the pancreas or bile duct is operator dependent, whereas aspiration of bile or pancreatic juice for miRNA analysis could be performed by all ERCP providers. ERCP is often indicated in the setting of suspected PDAC for the palliation of obstructive jaundice or an indeterminate pancreatic duct stricture, so aspiration of bile or pancreatic juice may enhance the diagnostic performance of this intervention.

Analysis of miRNA expression from surgical explants indicates that an miRNA panel may even be able to distinguish cholangiocarcinoma from PDAC, which will be increasingly important as systemic therapies are personalized for these cancer sub-types. [49] Impressively, the performance of plasma alone had excellent accuracy for distinguishing PDAC from CP and controls. Nonetheless, future studies should explore the potential complementary roles of plasma+bile miRNA as biomarkers for differentiating pancreatic cancer from cholangiocarcinoma or metastatic lesions to the pancreas.

Peripheral miRNA as Prognostic Markers

miR-10b, miR-21, and miR-155 are important in PDAC biology.[12, 13, 15, 16, 19] However, miR-21 did not serve as a good plasma, bile, or pancreatic juice biomarker. This may be because the mechanisms regulating miRNA release into the circulation are complex, cannot be generalized to all miRNAs, and have not been clearly delineated. Moreover, using in situ hybridization (ISH) with specific 5′ fluorescein-conjugated locked nucleic acid-modified probes, miR-10b and miR-21 expression are abundant in PDAC cells and are also present in cancer-associated fibroblasts were previously demonstrated; on the other hand, miR-155 localizes to CD45+ T cells within the pancreatic tumor microenvironment and is not present in cancer cells.[19, 50] Therefore, the cell type in which a particular miRNA is expressed in PDAC does not necessarily dictate its usefulness as a peripheral biomarker. It should also be noted that miRNAs can be packaged in microparticles such as exosomes prior to their release into the circulation or other bodily fluids; [47] these possibilities were not explored.

Significant differences in the expression of miRNA among PDAC individuals with stage I-III versus stage 1V disease. However, this may be a type II error given our limited sample size (n=70) was not observed. Moreover, the cross-sectional study did not include serial collection of plasma to track miRNA expression following surgical resection or systemic therapy, and PDAC patient follow-up was relatively short. Therefore, one cannot extrapolate whether specific miRNA differentially expressed in plasma, bile or pancreatic juice will correlate with response to specific therapies or survival. Nonetheless, the present findings indicate that a plasma miRNA signature may serve as a non-invasive diagnostic test for PDAC, and that obtaining bile or pancreatic juice for miRNA during ERCP for the evaluation and treatment of pancreatobiliary strictures may be equally accurate. Moreover, the current miRNA signature could complement recently described protein biomarkers[51] without being influenced by jaundice or age. Age- and sex-matched individuals with CP and controls in the training cohort to eliminate the potential for these covariates to influence the results were deliberately chosen. Significant differences in plasma miRNA expression among control subjects with increasing age were not observed. [47, 52] The validation cohort represented a real-world cohort of individuals presenting for EUS, ERCP, or both with suspected PDAC, CP, and choledocholithiasis (controls); plasma and bile Panels applied to this validation group had excellent sensitivity and specificity for diagnosing PDAC. Other supporting evidence regarding specific miRNAs that can be used to diagnose and treat particular forms of pancreatic cancer can be found in U.S. provisional patent application No. 61/973,144 field on Mar. 31, 2014 which is incorporated herein by reference in its entirety.

Effect of Surgery on miR-10b Levels

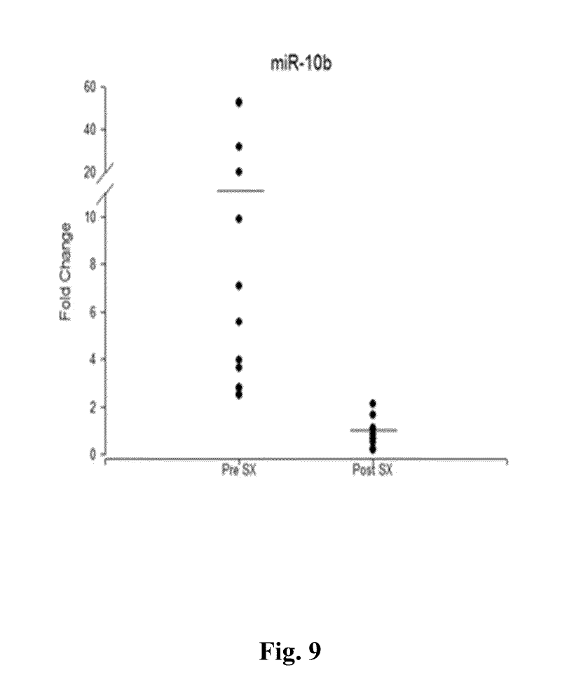

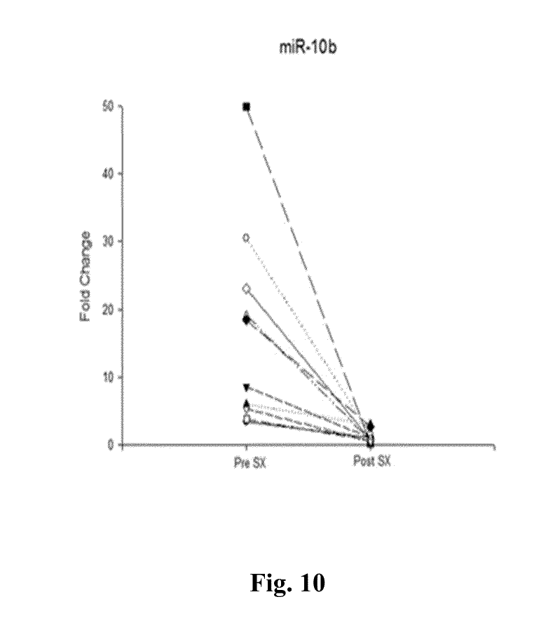

Referring now to FIGS. 9 and 10. Plasma levels for miR-10b were elevated in 10 consecutive patients diagnosed with pancreatic cancer whose blood was collected prospectively, ranging from a 2.5 fold to a 50-fold increase by comparison with normal miR-10b levels in individuals without pancreatic cancer (FIG. 9). Importantly, one day following surgical removal of the pancreatic cancer, the mean miR-10b plasma level was dramatically and significantly decreased in these patients (FIG. 10). Moreover, removal of the cancer resulted in lower plasma levels of miR-10b in each of the 10 patients.

These observations provide additional evidence that miR-10b is a marker for pancreatic cancer. These data indicate that an increase in the levels in miR-10b is due to release of this microRNA from the pancreatic tumor. The results also suggest that miR-10 could serve as a marker for pancreatic cancer recurrence.

### REFERENCES

1 Esquela-Kerscher A, Slack F J. Oncomirs—microRNAs with a role in cancer. Nature reviews Cancer 2006; 6:259-69.
2 Iorio M V, Croce C M. MicroRNAs in cancer: small molecules with a huge impact. Journal of clinical oncology: official journal of the American Society of Clinical Oncology 2009; 27:5848-56.
3 Valencia-Sanchez M A, Liu J, Hannon G J, Parker R. Control of translation and mRNA degradation by miRNAs and siRNAs. Genes & development 2006; 20:515-24.
4 Iorio M V, Croce C M. MicroRNA dysregulation in cancer: diagnostics, monitoring and therapeutics. A comprehensive review. EMBO molecular medicine 2012; 4:143-59.
5 Stahlhut C, Slack F J. MicroRNAs and the cancer phenotype: profiling, signatures and clinical implications. Genome medicine 2013; 5:111.
6 Croce C M. Causes and consequences of microRNA dysregulation in cancer. Nature reviews Genetics 2009; 10:704-14.
7 Hoffman A E, Liu R, Fu A, Zheng T, Slack F, Zhu Y. Targetome profiling, pathway analysis and genetic association study implicate miR-202 in lymphomagenesis. Cancer epidemiology, biomarkers & prevention: a publication of the American Association for Cancer Research, cosponsored by the American Society of Preventive Oncology 2013; 22:327-36.
8 Pizzini S, Bisognin A, Mandruzzato S, Biasiolo M, Facciolli A, Perini L, et al. Impact of microRNAs on regulatory networks and pathways in human colorectal carcinogenesis and development of metastasis. BMC genomics 2013; 14:589.
9 Xia H, Hui K M. MicroRNAs involved in regulating epithelial-mesenchymal transition and cancer stem cells as molecular targets for cancer therapeutics. Cancer gene therapy 2012; 19:723-30.
10 Frampton A E, Castellano L, Colombo T, Giovannetti E, Krell J, Jacob J, et al. MicroRNAs cooperatively inhibit a network of tumor suppressor genes to promote pancreatic tumor growth and progression. Gastroenterology 2014; 146:268-77 e18.
11 Mohr A M, Bailey J M, Lewallen M E, Liu X, Radhakrishnan P, Yu F, et al. MUC1 regulates expression of multiple microRNAs involved in pancreatic tumor progression, including the miR-200c/141 cluster. PloS one 2013; 8:e73306.
12 Ouyang H, Gore J, Deitz S, Korc M. microRNA-10b enhances pancreatic cancer cell invasion by suppressing TIP30 expression and promoting EGF and TGF-beta actions. Oncogene 2013.
13 Szafranska A E, Davison T S, John J, Cannon T, Sipos B, Maghnouj A, et al. MicroRNA expression alterations are linked to tumorigenesis and non-neoplastic processes in pancreatic ductal adenocarcinoma. Oncogene 2007; 26:4442-52.
14 Xu L, Li Q, Xu D, Wang Q, An Y, Du Q, et al. hsa-miR-141 downregulates TM4SF1 to inhibit pancreatic cancer cell invasion and migration. International journal of oncology 2014; 44:459-66.
15 Greither T, Grochola L F, Udelnow A, Lautenschlager C, Wurl P, Taubert H. Elevated expression of microRNAs 155, 203, 210 and 222 in pancreatic tumors is associated with poorer survival. International journal of cancer Journal international du cancer 2010; 126:73-80.
16 Bloomston M, Frankel W L, Petrocca F, Volinia S, Alder H, Hagan J P, et al. MicroRNA expression patterns to differentiate pancreatic adenocarcinoma from normal pancreas and chronic pancreatitis. JAMA: the journal of the American Medical Association 2007; 297:1901-8.
17 Ma M Z, Kong X, Weng M Z, Cheng K, Gong W, Quan Z W, et al. Candidate microRNA biomarkers of pancreatic ductal adenocarcinoma: meta-analysis, experimental validation and clinical significance. Journal of experimental & clinical cancer research: CR 2013; 32:71.
18 Sarner M, Cotton P B. Classification of pancreatitis. Gut 1984; 25:756-9.
19 Preis M, Gardner T B, Gordon S R, Pipas J M, Mackenzie T A, Klein E E, et al. MicroRNA-10b expression correlates with response to neoadjuvant therapy and survival in pancreatic ductal adenocarcinoma. Clinical cancer research: an official journal of the American Association for Cancer Research 2011; 17:5812-21.
20 Szafranska A E, Doleshal M, Edmunds H S, Gordon S, Luttges J, Munding J B, et al. Analysis of microRNAs in pancreatic fine-needle aspirates can classify benign and malignant tissues. Clinical chemistry 2008; 54:1716-24.
21 Sicard F, Gayral M, Lulka H, Buscail L, Cordelier P. Targeting miR-21 for the therapy of pancreatic cancer. Molecular therapy: the journal of the American Society of Gene Therapy 2013; 21:986-94.
22 Park J K, Henry J C, Jiang J, Esau C, Gusev Y, Lerner M R, et al. miR-132 and miR-212 are increased in pancreatic cancer and target the retinoblastoma tumor suppressor. Biochemical and biophysical research communications 2011; 406:518-23.
23 Gore A J, Deitz S L, Palam L R, Craven K E, Korc M. Pancreatic cancer-associated retinoblastoma 1 dysfunction enables TGF-beta to promote proliferation. The Journal of clinical investigation 2014; 124:338-52.
24 Cai K, Wang Y, Bao X. MiR-106b promotes cell proliferation via targeting RB in laryngeal carcinoma. Journal of experimental & clinical cancer research: CR 2011; 30:73.
25 Ivanovska I, Ball A S, Diaz R L, Magnus J F, Kibukawa M, Schelter J M, et al. MicroRNAs in the miR-106b family regulate p21/CDKN1A and promote cell cycle progression. Molecular and cellular biology 2008; 28:2167-74.
26 Garofalo M, Romano G, Di Leva G, Nuovo G, Jeon Y J, Ngankeu A, et al. EGFR and MET receptor tyrosine kinase-altered microRNA expression induces tumorigenesis and gefitinib resistance in lung cancers. Nature medicine 2012; 18:74-82.
27 Preis M, Korc M. Signaling pathways in pancreatic cancer. Critical reviews in eukaryotic gene expression 2011; 21:115-29.
28 Liu J, Gao J, Du Y, Li Z, Ren Y, Gu J, et al. Combination of plasma microRNAs with serum CA19-9 for early detection of pancreatic cancer. International journal of cancer Journal international du cancer 2012; 131:683-91.
29 Takiuchi D, Eguchi H, Nagano H, Iwagami Y, Tomimaru Y, Wada H, et al. Involvement of microRNA-181b in the gemcitabine resistance of pancreatic cancer cells. Pancreatology: official journal of the International Association of Pancreatology 2013; 13:517-23.
30 Chang K H, Mestdagh P, Vandesompele J, Kerin M J, Miller N. MicroRNA expression profiling to identify and validate reference genes for relative quantification in colorectal cancer. BMC cancer 2010; 10:173.
31 Livak K J, Schmittgen T D. Analysis of relative gene expression data using real-time quantitative PCR and the 2(-Delta Delta C(T)) Method. Methods 2001; 25:402-8.
32 Dunn O J. Multiple Comparisons Among Means. Journal of the American Statistical Association 1961; 56:52-64.
33 Siegel R, Naishadham D, Jemal A. Cancer statistics, 2013. CA: a cancer journal for clinicians 2013; 63:11-30.
34 Conroy T, Desseigne F, Ychou M, Bouche O, Guimbaud R, Becouarn Y, et al. FOLFIRINOX versus gemcitabine for metastatic pancreatic cancer. The New England journal of medicine 2011; 364:1817-25.
35 Von Hoff D D, Ervin T, Arena F P, Chiorean E G, Infante J, Moore M, et al. Increased survival in pancreatic cancer with nab-paclitaxel plus gemcitabine. The New England journal of medicine 2013; 369:1691-703.
36 Lee L S, Szafranska-Schwarzbach A E, Wylie D, Doyle L A, Bellizzi A M, Kadiyala V, et al. Investigating MicroRNA Expression Profiles in Pancreatic Cystic Neoplasms. Clinical and translational gastroenterology 2014; 5:e47.
37 Matthaei H, Wylie D, Lloyd M B, Dal Molin M, Kemppainen J, Mayo S C, et al. miRNA biomarkers in cyst fluid augment the diagnosis and management of pancreatic cysts. Clinical cancer research: an official journal of the American Association for Cancer Research 2012; 18:4713-24.
38 Yu J, Li A, Hong S M, Hruban R H, Goggins M. MicroRNA alterations of pancreatic intraepithelial neoplasias. Clinical cancer research: an official journal of the American Association for Cancer Research 2012; 18:981-92.
39 Bauer A S, Keller A, Costello E, Greenhalf W, Bier M, Borries A, et al. Diagnosis of pancreatic ductal adenocarcinoma and chronic pancreatitis by measurement of microRNA abundance in blood and tissue. PloS one 2012; 7:e34151.
40 Schultz N A, Dehlendorff C, Jensen B V, Bjerregaard J K, Nielsen K R, Bojesen S E, et al. MicroRNA biomarkers in whole blood for detection of pancreatic cancer. JAMA: the journal of the American Medical Association 2014; 311:392-404.
41 Liu R, Chen X, Du Y, Yao W, Shen L, Wang C, et al. Serum microRNA expression profile as a biomarker in the diagnosis and prognosis of pancreatic cancer. Clinical chemistry 2012; 58:610-8.
42 Wang J, Chen J, Chang P, LeBlanc A, Li D, Abbruzzesse J L, et al. MicroRNAs in plasma of pancreatic ductal adenocarcinoma patients as novel blood-based biomarkers of disease. Cancer prevention research 2009; 2:807-13.
43 Buchsbaum D J, Croce C M. Will detection of microRNA biomarkers in blood improve the diagnosis and survival of patients with pancreatic cancer? JAMA: the journal of the American Medical Association 2014; 311:363-5.
44 Locker G Y, Hamilton S, Harris J, Jessup J M, Kemeny N, Macdonald J S, et al. ASCO 2006 update of recommendations for the use of tumor markers in gastrointestinal cancer. Journal of clinical oncology: official journal of the American Society of Clinical Oncology 2006; 24:5313-27.
45 Chen X, Ba Y, Ma L, Cai X, Yin Y, Wang K, et al. Characterization of microRNAs in serum: a novel class of biomarkers for diagnosis of cancer and other diseases. Cell research 2008; 18:997-1006.
46 Lawrie C H, Gal S, Dunlop H M, Pushkaran B, Liggins A P, Pulford K, et al. Detection of elevated levels of tumour-associated microRNAs in serum of patients with diffuse large B-cell lymphoma. British journal of haematology 2008; 141:672-5.
47 Creemers E E, Tijsen A J, Pinto Y M. Circulating microRNAs: novel biomarkers and extracellular communicators in cardiovascular disease? Circulation research 2012; 110:483-95.
48 Cote G A, Sherman S. Biliary stricture and negative cytology: what next? Clinical gastroenterology and hepatology: the official clinical practice journal of the American Gastroenterological Association 2011; 9:739-43.
49 Collins A L, Wojcik S, Liu J, Frankel W L, Alder H, Yu L, et al. A Differential MicroRNA Profile Distinguishes Cholangiocarcinoma from Pancreatic Adenocarcinoma Annals of surgical oncology 2013.
50 Sempere L F, Preis M, Yezefski T, Ouyang H, Suriawinata A A, Silahtaroglu A, et al. Fluorescence-based codetection with protein markers reveals distinct cellular compartments for altered MicroRNA expression in solid tumors. Clinical cancer research: an official journal of the American Association for Cancer Research 2010; 16:4246-55.
51 Nie S, Lo A, Wu J, Zhu J, Tan Z, Simeone D M, et al. Glycoprotein Biomarker Panel for Pancreatic Cancer Discovered by Quantitative Proteomics Analysis. Journal of proteome research 2014.
52 Noren Hooten N, Fitzpatrick M, Wood W H, 3rd, De S, Ejiogu N, Zhang Y, et al. Age-related changes in microRNA levels in serum. Aging 2013; 5:725-40.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

We claim:

1. A method for treating a patient, comprising the steps of:
contacting a portion of plasma from a patient with at least one probe specific for at least one miRNA selected from the group consisting of miR-10b, and miR-106b;
using a signal produced by the contacting step to determine a level of at least one miRNA selected from the group consisting of: miR-10b, and miR-106b, in the sample;
comparing the level of the at least one miRNA measured in the sample to a statistically validated threshold for miR-10b, and/or miR-106b, levels; and
concluding that the patient is positive for pancreatic ductal adenocarcinoma if the threshold for miR-10b in the sample is greater than about 3.579, or the threshold of miR-106b in the sample is greater than about 2.920.

2. The method according to claim 1, wherein the contacting step further includes contacting the sample with at least one probe for at least one miRNA, selected from the group consisting of: miR-21, miR-30c, miR-132, miR-155, miR-181a, miR-181b, miR-196a, and miR-212.

3. The method according to claim 1, wherein the patient is concluded to be positive for pancreatic ductal adenocarcinoma if the threshold of miR-10b measured in the sample is greater than about 3.579, and the threshold of miR-106b measured in the sample is greater than about 2.920.

4. The method according to claim 1, wherein the contacting step includes generating cDNA from RNA present in the sample.

5. The method according to claim 2, wherein the contacting step includes generating cDNA from RNA present in the sample.

6. The method according to claim 1, further including the step of:
recommending that the patient be treated for pancreatic ductal adenocarcinoma if the concluding step is positive for pancreatic ductal adenocarcinoma.

7. A method for treating a patient, comprising the steps of:
contacting a portion of bile from a patient with at least one probe specific for at least one miRNA selected from the group consisting of miR-10b, miR-106b, miR-155, and miR-212;
using a signal produced by the contacting step to determine a level of at least one miRNA selected from the group consisting of: miR-10b, miR-106b, miR-155, and miR-212 in the sample;
comparing the level of the at least one miRNA measured in the sample to a statistically validated threshold for miR-10b, miR-106b, miR-155, and/or miR-212 levels; and
concluding that the patient is positive for pancreatic ductal adenocarcinoma if the threshold of miR-10b in the sample is greater than about 3.497, or the threshold of miR-106b in the sample is greater than about 5.261, or the threshold of miR-155 in the sample is greater than about 5.232, or the threshold of miR-212 in the sample is greater than about 4.163.

8. The method according to claim 7, wherein the contacting step further includes contacting the sample with at least one probe for at least one miRNA, selected from the group consisting of: miR-21, miR-30c, miR-132, miR-181a, miR-181b, and miR-196a.

9. The method according to claim 7, wherein the patient is concluded to be positive for pancreatic ductal adenocarcinoma if the threshold of miR-10b in the sample is greater than about 3.497, the threshold of miR-106b in the sample is greater than about 5.261, the threshold of miR-155 in the sample is greater than about 5.232, and the threshold of miR-212 in the sample is greater than about 4.163.

10. The method according to claim 7, wherein the contacting step includes generating cDNA from RNA present in the sample.

11. The method according to claim 8, wherein the contacting step includes generating cDNA from RNA present in the sample.

12. The method according to claim 7, further including the step of:
recommending that the patient be treated for pancreatic ductal adenocarcinoma if the concluding step is positive for pancreatic ductal adenocarcinoma.

13. A method for treating a patient, comprising the steps of:
contacting a portion of pancreatic juice from a patient with at least one probe specific for at least one miRNA selected from the group consisting of miR-155, miR-106b, miR-10b, miR-212, and miR-30C;
using a signal produced by the contacting step to determine a level of at least one miRNA selected from the group consisting of: miR-155, miR-106b, miR-10b, miR-212, and miR-30C in the sample;
comparing the level of the at least one miRNA measured in the sample to a statistically validated threshold for miR-155, miR-106b, miR-10b, miR-212, and/or miR-30C levels; and
concluding that the patient is positive for pancreatic ductal adenocarcinoma if the threshold of miR-155 in the sample is greater than about 4.42, or the threshold of miR-106b in the sample is greater than about 4.54, or the threshold of miR-10b in the sample is greater than about 4.54, or the threshold of miR-212 in the sample is greater than about 3.69 or the threshold of miR-30 measured in the sample is greater than 3.27.

14. The method according to claim 13, wherein the contacting step further includes contacting the sample with at least one probe for at least one miRNA, selected from the group consisting of: miR-21, miR-132, miR-181a, miR-181b, and miR-196a.

15. The method according to claim 13, wherein the patient is concluded to be positive for pancreatic ductal adenocarcinoma if the threshold of miR-155 in the sample is greater than about 4.42, the threshold of miR-106b in the sample is greater than about 4.54, the threshold of miR-10b in the sample is greater than about 4.54, the threshold of miR-212 in the sample is greater than about 3.69 and the threshold of miR-30 measured in the sample is greater than 3.27.

16. The method according to claim 13, wherein the contacting step includes generating cDNA from RNA present in the sample.

17. The method according to claim 14, wherein the contacting step includes generating cDNA from RNA present in the sample.

18. The method according to claim 13, further including the step of:
recommending that the patient be treated for pancreatic ductal adenocarcinoma if the concluding step is positive for pancreatic ductal adenocarcinoma.
